(19) United States
(12) Patent Application Publication
(10) Pub. No.: US 2022/0213178 A1
(43) Pub. Date: Jul. 7, 2022

(54) Enhanced Transfection

(71) Applicant: The University of Nottingham, Nottingham, Nottinghamshire (GB)

(72) Inventor: James Dixon, Nottingham, Nottinghamshire (GB)

(73) Assignee: The University of Nottingham, Nottingham, Nottinghamshire (GB)

(21) Appl. No.: 17/440,991

(22) PCT Filed: Mar. 23, 2020
(86) PCT No.: PCT/GB2020/050766

§ 371 Date: Sep. 20, 2021

(30) Foreign Application Priority Data
Mar. 21, 2019 (GB) 1903873.6

(51) Int. Cl. C07K 16/18; C07K 16/44

(52) U.S. Cl. CPC C07K 16/18; C07K 2317/569; C07K 2317/622; C07K 16/44

(57) ABSTRACT

The invention relates to a method of transfecting a cell with a molecule, the method comprising the steps of: adding a molecule for transfection to the cells; and modulating the activity of protein kinase C (PKC) in the cell and/or providing a pH responsive peptide comprising between about 5 and about 20 histidine residues; and related compositions and uses in therapy.

This invention relates to methods of cell transfection, associated reagents for transfection, and therapeutic applications of the method.

Gene delivery is a useful tool for investigating and manipulating cellular processes, and for therapy. Ex vivo genetic modification of human cells has been shown to significantly improve their therapeutic potentials. The most commonly exploited nucleic acid delivery vehicles, in both academic and clinical labs, are viral vectors. Viral vectors have been demonstrated to be highly efficient. However, there is the potential for random integration of the virus vector into the host genome, which may interrupt essential gene expression and cellular processes. Due to the safety concerns, high cost and technical difficulty of viral gene delivery, attention has turned to the development of non-viral gene delivery systems.

Non-viral gene delivery has relatively high efficiency in many cell lines, but stem cells and post-mitotic cells are known to be recalcitrant (0-35% transfection efficiency) with present non-viral delivery methods. One method developed to overcome the poor transfection efficiency of non-viral gene delivery is electroporation. Electroporation results in high transfection, but results in low cell viability post-transfection and scalability is a concern. Alternative methods, which all require specialised setups, include microinjection, gene gun, sonoporation, laser and cell deformation. At present, high transfection efficiency in hard-to-transfect cell types using non-viral gene delivery remains unsolved.

Another recently developed method of delivery has been disclosed by Dixon et al (PCT Publication No: WO2015092417, which is herein incorporated by reference). Disclosed is a method of transduction of cargo molecules into living cells, whereby a delivery molecule comprising a cargo, glycosaminoglycan (GAG) binding element (which is capable of binding to GAG on the surface of a cell), and a protein transduction domain provides efficient transduction of protein into a cell. A modification of this approach is provided in patent application publication no. WO2016207638 (which is incorporated herein by reference), which discloses a pH mediated cell delivery vehicle comprising a cargo or cargo-binding molecule for binding to a cargo, a protein transduction domain, and a GAG binding element, which is capable of binding to GAG on the surface of a cell, wherein the GAG binding element is a peptide which is modified to comprise one or more histidine residues which are capable of being protonated in an acidic environment. Despite advances in transfection methods, some cell types remain difficult to transfect. Therefore, there is a need for further improvement.

Looking to improve transfection of hard to transfect cells, Ho et al (2016) have demonstrated that chemical inhibition of histone deacetylases (HDACs) can enhance methods of non-viral transfection. This suggests that it is possible to modify cell activity to aid the transfection process.

It is also desirable and known that the above transfection methods can transfect molecules other than nucleic acid into cells, such as proteins and nanoparticles. Therefore, it is desirable to improve both nucleic acid delivery and other cargos, such as proteins, peptides and nanoparticles that would otherwise be difficult to pass across the cell membrane.

The aim of the invention is to provide improved transfection efficiency of cells, including difficult to transfect cells.

According to a first aspect of the invention, there is provided a method of transfecting a cell with a molecule, the method comprising the steps of:
adding a molecule for transfection to the cells; and
modulating the activity of protein kinase C (PKC) in the cell and/or providing a pH responsive peptide comprising between about 5 and about 20 histidine residues.

The histidine residues of the pH responsive peptide may be capable of being protonated in an acidic environment of an endosome.

The invention herein surprisingly finds that modulation of PKC activity in a cell, optionally with histone deacetylase (HDAC) modulation, has a significant effect in dramatically enhancing the transfection of cells, and advantageously the transfection of cell types that presently cannot be efficiently transfected with gold-standard reagents. In particular, the invention herein has found the effect of transfection efficiency for HDAC modulation alone is in the order of 3-10 fold increased efficiency, PKC modulation alone can provide a significantly greater 10-20 fold increase in efficiency, and HDAC modulation and PKC modulation together can provide a surprising 50-200, or more, fold increase in efficiency.

### PKC Modulation

Modulating the activity of PKC in the cell may comprise adding an agent to the cell to modulate the activity of PKC in the cell.

In one embodiment, the modulation of the activity of PKC in the cell comprises inhibition of PKC activity. The inhibition may comprise a complete (i.e. 100%) inhibition or a reduction in activity. In another embodiment, the inhibition may comprise a significant or substantial inhibition or a reduction in activity. The activity of the PKC in the cell may be inhibited by at least 5%. In another embodiment, the activity of the PKC in the cell may be inhibited by at least 10%. In another embodiment, the activity of the PKC in the cell may be inhibited by at least 20%. In another embodiment, the activity of the PKC in the cell may be inhibited by at least 50%. In another embodiment, the activity of the PKC in the cell may be inhibited by at least 80%. The skilled person will understand that the success or level of PKC inhibition may be determined by detecting the phosphorylation state of one or more PKC targets.

The activity of the PKC in the cell may be inhibited by an inhibitor of PKC. In one embodiment, modulating the activity of PKC in the cell comprises adding a modulator of PKC to the cell. The modulator of PKC may be a PKC inhibitor.

In one embodiment, the modulation of the activity of PKC in the cell comprises activation of PKC activity. The activation may comprise a significant or substantial activation or an increase in activity. The activity of the PKC in the cell may be increased by at least 5%. In another embodiment, the activity of the PKC in the cell may be increased by at least 10%. In another embodiment, the activity of the PKC in the cell may be increased by at least 20%. In another embodiment, the activity of the PKC in the cell may be increased by at least 50%. In another embodiment, the activity of the PKC in the cell may be increased by at least 80%. The skilled person will understand that the success or level of PKC activation may be determined by detecting the phosphorylation state of one or more PKC targets.

The activity of the PKC in the cell may be increased by an activator of PKC. In one embodiment, modulating the activity of PKC in the cell comprises adding a modulator of PKC to the cell. The modulator of PKC may be a PKC activator. In one embodiment, the modulation of the activity of PKC in the cell comprises upregulating expression of PKC in the cell. Upregulating expression may be achieved by providing a molecule capable of upregulating PKC. The molecule may be nucleic acid, such as siRNA. The molecule may be nucleic acid, such as DNA or RNA, encoding PKC for overexpression.

The modulator of PKC may be selected from the group comprising phorbol 12-myristate 13-acetate (PMA), Ingenol 3-angelate (I3A) and bryostatin. In one embodiment, the modulator of PKC comprises phorbol 12-myristate 13-acetate (PMA) (also known as 12-O-tetradecanoylphorbol 13-acetate (TPA)). In another embodiment, the modulator of PKC comprises Ingenol 3-angelate (I3A). In another embodiment, the modulator of PKC comprises bryostatin. The skilled person will recognise that functional analogues and derivatives of such modulators may also be used as an alternative. In another embodiment, the modulator of PKC may be a genetic silencer, such as siRNA, to inhibit expression of PKC isoforms, thereby reducing its activity.

In one embodiment, the modulator of PKC may be an inhibitor of PKC selected from the group comprising Bisindolylmaleimide I (otherwise known as 2-[1-(3-Dimethylaminopropyl)indol-3-yl]-3-(indol-3-yl) maleimide or GFX (GF109203X)), Calphostin C, and Go6976 (5,6,7,13-Tetrahydro-13-methyl-5-oxo-12H-indolo[2,3-a]pyrrolo[3,4-c]carbazole-12-propanenitrile); or combinations thereof.

In one embodiment, the modulation of the activity of PKC in the cell comprises down-regulating expression of PKC in the cell. Down-regulating expression may be achieved by providing a molecule capable of down-regulating PKC. The molecule may be nucleic acid, such as siRNA that is capable of supressing expression of PKC. The skilled person will be familiar with techniques to upregulate and down regulate specific proteins in a cell, such as PKC.

The modulator of PKC may be provided at an effective concentration and amount to provide the desired modulation, such as activation. The skilled person will recognise that different PKC modulating agents may require different concentrations and amounts for effective modulation of PKC. For example, the amount of PKC modulator may be between 10 nm and about 100 μM. In another embodiment, the amount of PKC modulator may be between 0.1 and 100 μM. In another embodiment, the amount of PKC modulator may be between 10 nm and 1 μM. In another embodiment, the amount of PKC modulator may be between 0.1 and 1 μM.

Modulation of the activity of PKC, for example by adding an agent for PKC activity modulation, may be concurrently with, or after, the addition of the molecule for transfection to the cells. Without being bound by theory, it is understood that PKC modulation is important to endosome release of the molecule for transfection. Therefore, the PKC modulation may be at a suitable time to enhance endosome release into the cell.

The modulation, such as inhibition, of PKC activity may be temporary. For example, the modulation may occur for sufficient time to aid the transfection process. The modulation, such as activation, of PKC activity may be for 4 hours or less. The modulation, such as activation, of PKC activity may be after starting the transfection. The modulation, such as activation, of PKC activity may up to 3 hours after starting the transfection. In one embodiment, the modulation, such as activation, of PKC activity may be at a time of between about 1 and about 3 hours after starting transfection. In another embodiment, the modulation, such as activation, of PKC activity may be at a time of between about 12 and about 24 hours after starting transfection. In another embodiment, the modulation, such as activation, of PKC activity may be at a time of between about 0 and about 24 hours after starting transfection.

### HDAC Modulation

The method of transfecting a cell with a molecule may further comprise the modulation of histone deacetylase (HDAC) in the cell. In one embodiment, the modulation of the activity of HDAC in the cell comprises inhibition of HDAC activity. The modulation, such as inhibition, of HDAC in the cell may be concurrently with the modulation of, such as activation of, the activity of PKC in the cell. In one embodiment HDAC is inhibited and PKC is activated. In another embodiment HDAC is inhibited and PKC is inhibited. In another embodiment HDAC is activated and PKC is activated. In another embodiment HDAC is activated and PKC is inhibited.

Advantageously several HDAC inhibitors (HDACi) with more specific or broader specificity have all shown a level of transfection promoting activity without toxicity in multiple cell lines.

The HDAC inhibition may comprise a complete (i.e. 100%) inhibition or a reduction in activity of HDAC. In another embodiment, the inhibition may comprise a significant or substantial inhibition or a reduction in activity of HDAC. The activity of the HDAC in the cell may be inhibited by at least 5%. In another embodiment, the activity of the HDAC in the cell may be inhibited by at least 10%. In another embodiment, the activity of the HDAC in the cell may be inhibited by at least 20%. In another embodiment, the activity of the HDAC in the cell may be inhibited by at least 50%. In another embodiment, the activity of the HDAC in the cell may be inhibited by at least 80%. The skilled person will understand that the success or level of HDAC inhibition may be determined by an assay, such as a histone deacetylase assay (for example the Histone Deacetylase Assay Kit, Fluorometric, by Sigma-Aldrich/Merck, or equivalent assays and kits thereof).

The inhibition of HDAC may comprise the use of a HDAC inhibitor. Therefore, in one embodiment, the method of transfecting a cell with a molecule may further comprise the addition of a HDAC inhibitor during or prior to transfection with the molecule.

The HDAC inhibitor may be selected from the group comprising suberoylanilide hydroxamic acid (SAHA), panobinostat, trichostatin A (TSA), tubastatin A, and valproic acid. In one embodiment, the HDAC inhibitor comprises suberoylanilide hydroxamic acid (SAHA). In another embodiment, the HDAC inhibitor comprises panobinostat, trichostatin A (TSA). In another embodiment, the HDAC inhibitor comprises tubastatin A. In another embodiment, the HDAC inhibitor comprises valproic acid. The skilled person will recognise that functional analogues and derivatives of such inhibitors may also be used as an alternative. In another embodiment, the HDAC inhibitor may be a genetic silencer, such as siRNA, to inhibit expression of HDAC, thereby reducing its activity.

In one embodiment, the modulation of the activity of HDAC in the cell comprises enhancing HDAC activity, for example by providing an HDAC enhancer. The HDAC enhancer may comprise N-acetylthiourea. Additionally or alternatively, HDAC inhibitors may be inhibited in order to enhance HDAC activity. For example HDAC inhibitor TSA may be inhibited. The TSA inhibitor may comprise ISTA1.

In one embodiment, the modulation of the activity of HDAC in the cell comprises upregulating expression of HDAC in the cell. Upregulating expression may be achieved by providing a molecule capable of upregulating HDAC. The molecule may be nucleic acid, such as siRNA. The molecule may be nucleic acid, such as DNA or RNA, encoding HDAC for expression, such as overexpression. The molecule to enhance HDAC activity may comprise a vector encoding HDAC and optionally a promoter to drive the expression. The skilled person will be familiar with techniques to upregulate and down regulate specific proteins in a cell, such as HDAC.

A combination of PKC and HDAC inhibition may comprise the use of PMA/TPA in combination with one or more of panobinostat, TSA or SAHA.

In an embodiment wherein the cells to be transfected are T- or B-cells, or a hybrid thereof, the PKC modulator may comprise PMA/TPA. In an embodiment wherein the cells to be transfected are 2 hybrid human T/B cells the PKC modulator may comprise PMA/TPA.

In an embodiment wherein the cells to be transfected are macrophage cells, the PKC modulator may comprise PMA/TPA, which may further be used together with the HDAC inhibitor SAHA. In an embodiment wherein the cells to be transfected are lymphoblastoid cell line cells (LCL), the PKC modulator may comprise PMA/TPA, which may further be used together with the HDAC inhibitor SAHA. The LCL cells may be lymphoblastoid human B cells.

The modulator of HDAC may be provided at an effective concentration and amount to provide the desired inhibition of HDAC activity. The skilled person will recognise that different HDAC inhibitor agents may require different concentrations and amounts for effective inhibition of HDAC. For example, the amount of HDAC inhibitor may be between 10 nM and about 100 μM. In another embodiment, the amount of HDAC modulator may be between 0.1 and 100 μM. In another embodiment, the amount of HDAC modulator may be between 0.1 and 10 μM. In another embodiment, the amount of HDAC modulator may be between 1 and 10 μM.

The HDAC inhibition may be temporary. For example, the inhibition may occur for sufficient time to aid the transfection process. The HDAC inhibition may be for 4 hours or less. The HDAC inhibition may be after starting the transfection. The HDAC inhibition may up to 6 hours after starting the transfection. In one embodiment, the HDAC inhibition may be at a time of between about 1 and about 6 hours after starting transfection.

In one embodiment, the HDAC inhibition and PKC modulation, such as inhibition, may be substantially concurrent with each other.

### Molecule for Transfection

The molecule for transfection may not be in itself capable of modulating the activity of protein kinase C (PKC) in the cell. The molecule for transfection may not comprise or consist of a protein kinase C (PKC) modulator, such as an inhibitor or activator.

The molecule for transfection may comprise nucleic acid, such as a nucleic acid vector. The molecule for transfection may comprise oligonucleotide. The molecule for transfection may comprise any of the group selected from siRNA, modified messenger RNAs (mRNAs), micro RNAs, DNA, PNA, LNA or constructs thereof. In one embodiment, the molecule for transfection is DNA. The nucleic acid, such as DNA, coding or non-coding for a protein or peptide. The nucleic acid, such as DNA, may comprise one or more gene sequences and/or one or more regulatory sequences.

In another embodiment, the molecule for transfection may comprise a protein. The molecule for transfection may comprise a peptide. The molecule for transfection may comprise a physiologically or metabolically relevant protein. The molecule for transfection may comprise an intracellular protein. The molecule for transfection may comprise a signal protein, which is a protein involved in a signal pathway. The molecule for transfection may comprise a protein involved with regulation of expression or metabolism of a cell. The molecule for transfection may comprise a protein involved with cell division. The molecule for transfection may comprise a protein involved with cell differentiation, such as stem cell differentiation. The molecule for transfection may comprise a protein required for induction of pluripotent stem cells. The molecule for transfection may comprise a protein involved with cardiac cell differentiation. The molecule for transfection may comprise a marker, such as a protein marker. The molecule for transfection may comprise a bacterial, or bacterially derived protein. The molecule for transfection may comprise a mammalian, or mammalian derived protein. The molecule for transfection may be any peptide, polypeptide or protein. The molecule for transfection may comprise research, diagnostic or therapeutic molecules. The molecule for transfection may comprise a transcription modulator, a member of signal production. The molecule for transfection may comprise an enzyme or substrate thereof, a protease, an enzyme activity modulator, a perturbimer and peptide aptamer, an antibody, a modulator of protein-protein interaction, a growth factor, or a differentiation factor.

The molecule for transfection may be a pre-protein. For example, excision domains may be provided in the delivery molecule, which is arranged to be cleaved upon entry or after entry into the cell. The molecule for transfection may be a protein arranged to be post-translationally modified within the cell. The molecule for transfection may be arranged to be functional once inside the cell. For example, the molecule for transfection may not be functional until after transfection into the cell.

The molecule for transfection may comprise any intracellular molecule. The molecule for transfection may comprise any protein or molecule having an intracellular function (mode of action), intracellular receptor, intracellular ligand, or intracellular substrate. The molecule for transfection may comprise a protein or molecule that is naturally/normally internalised into a cell. The molecule for transfection may comprise a protein intended for delivery or display in the cell surface, such as a cell surface receptor. The molecule for transfection may be selected from any of the group comprising a therapeutic molecule; a drug; a pro-drug; a functional protein or peptide, such as an enzyme or a transcription factor; a microbial protein or peptide; and a toxin; or nucleic acid encoding thereof.

In one embodiment, the molecule for transfection may comprise a transcription factor, or a nucleic acid encoding a transcription factor. Additionally or alternatively, the molecule for transfection may comprise a growth factor or a nucleic acid encoding a growth factor.

The molecule for transfection may be selected from any of the group comprising toxin, hormone transcription factors, such as jun, fos, max, mad, serum response factor (SRF), AP-1, AP2, myb, MyoD, myogenin, ETS-box containing proteins, TFE3, E2F, ATF1, ATF2, ATF3, ATF4, ZF5, NFAT, CREB, HNF4, C/EBP, SP1, CCAAT-box binding proteins, interferon regulation factor (IRF-1), Wilms tumor protein, ETS-binding protein, STAT, GATA-box binding proteins, e.g., GATA-3, transcription factor, such as HIF1a and RUNT, the forkhead family of winged helix proteins, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor VIII, factor IX, cystathione beta-synthase, branched chain ketoacid decarboxylase, isovaleryl-coA dehydrogenase, propionyl CoA carboxylase, methyl malonyl CoA mutase, glutaryl CoA dehydrogenase, beta-glucosidase, pyruvate carboxylate, hepatic phosphorylase, phosphorylase kinase, glycine decarboxylase, H-protein, T-protein, a cystic fibrosis transmembrane regulator (CFTR) sequence, a dystrophin cDNA sequence, Oct-3/4 (Pou5f1), Sox2, c-Myc, Klf4, RPE65 Nanog, and SoxB1; or fragments thereof, and/or combinations thereof.

The growth factor may comprise a growth factor selected from the group comprising adrenomedullin (AM); angiopoietin (Ang); autocrine motility factor; bone morphogenetic protein (BMP); ciliary neurotrophic factor (CNTF); Leukemia inhibitory factor (LIF); interleukin-6 (IL-6); colony-stimulating factor; macrophage colony-stimulating factor (M-CSF); granulocyte colony-stimulating factor (G-CSF); granulocyte macrophage colony-stimulating factor (GM-CSF); epidermal growth factor (EGF); ephrin; erythropoietin (EPO); fibroblast growth factor (FGF); glial cell line-derived neurotrophic factor (GDNF); neurturin; persephin; artemin; growth differentiation factor-9 (GDF9); hepatocyte growth factor (HGF); hepatoma-derived growth factor (HDGF); insulin; insulin-like growth factor; interleukin; keratinocyte growth factor (KGF); migration-stimulating factor (MSF); macrophage-stimulating protein (MSP), also known as hepatocyte growth factor-like protein (HGFLP); myostatin (GDF-8); neuregulin; neurotrophin; brain-derived neurotrophic factor (BDNF); nerve growth factor (NGF); neurotrophin; placental growth factor (PGF); platelet-derived growth factor (PDGF); renalase (RNLS); anti-apoptotic survival factor; T-cell growth factor (TCGF); thrombopoietin (TPO); transforming growth factor; transforming growth factor alpha (TGF-α); transforming growth factor beta (TGF-β); tumor necrosis factor-alpha (TNF-α); vascular endothelial growth factor (VEGF); and Wnt, or combinations thereof, and/or nucleic acid encoding such growth factors.

The nucleic acid to be transfected may upregulate, or may be capable of upregulating, a growth factor in the cells. In one embodiment, the nucleic acid to be transfected upregulates, or is capable of upregulating, BMP2 and/or VEGF expression in the cells.

The nucleic acid to be transfected may encode a transcription factor. The nucleic acid to be transfected may encode a BMP2 and/or VEGF.

In one embodiment, the molecule for transfection may comprise a complex of proteins, such as viral particle or a virus-like particle (VLP). The viral particle or a virus-like particle (VLP) may further comprise nucleic acid.

In one embodiment, the molecule for transfection may comprise a nanoparticle, such as a metal nanoparticle or polymer nanoparticle. The nanoparticle may be a rod, such as a metal rod. The nano-particle may be porous. The molecule for transfection may comprise a nano-structure. The molecule for transfection may comprise a superparamagnetic iron oxide nanoparticle (SPION). The molecule for transfection may comprise a non-small molecule.

The molecule for transfection may comprise non-covalently bound complexes such as protein-protein complexes, protein-mRNA, protein-non-coding RNA, protein-lipid and protein-small molecule complexes. Examples of such complexes are RNA induced silencing complexes (RISCs) and spliceosomes.

The molecule for transfection may have a molecular weight of at least 1 KDa. The molecule for transfection may have a molecular weight of at least 5 KDa. The molecule for transfection may have a molecular weight of at least 10 KDa. The molecule for transfection may have a molecular weight of at least 20 KDa. The molecule for transfection may have a molecular weight of 400 KDa or less. The molecule for transfection may have a molecular weight of 300 KDa or less. The molecule for transfection may have a molecular weight of between about 0.5 KDa and about 400 kDa. The molecule for transfection may have a molecular weight of between about 1 KDa and about 400 kDa. The molecule for transfection may have a molecular weight of between about 0.5 KDa and about 200 kDa. The molecule for transfection may have a molecular weight of between about 1 KDa and about 200 kDa. The molecule for transfection may have a molecular weight of between about 2 KDa and about 300 kDa. The molecule for transfection may have a molecular weight of between about 20 KDa and about 300 kDa. The molecule for transfection may have a molecular weight of between about 20 KDa and about 100 kDa.

Where the molecule for transfection comprises amino acids, the molecule for transfection may be between about 20 and about 30,000 amino acids in length. The molecule for transfection may be between about 20 and about 10,000 amino acids in length. The molecule for transfection may be between about 20 and about 5,000 amino acids in length. The molecule for transfection may be between about 20 and about 1000 amino acids in length. The molecule for transfection may be at least about 20 amino acids in length. The molecule for transfection may be at least about 100 amino acids in length.

In one embodiment, the molecule for transfection is associated with, complexed with, entrapped within, or linked to, a transfection delivery molecule. In one embodiment, the molecule for transfection is a cargo of a transfection delivery molecule. The molecule for transfection (i.e. the cargo) may be capable of binding, such as ionic or covalent binding, to a transfection delivery molecule. The molecule for transfection (i.e. cargo) may comprise an element for binding to a transfection delivery molecule. The molecule for transfection may comprise biotin, or alternatively streptavidin. The molecule for transfection may be biotinylated. The molecule for transfection may comprise an affinity tag capable of binding to a complementary affinity tag on a transfection delivery molecule.

The molecule for transfection may be a fusion peptide comprising the molecule for transfection and the transfection delivery molecule.

The terms “molecule for transfection” and “cargo” may herein be used interchangeably.

### Transfection Delivery Molecule

The transfection delivery molecule may be selected from the group comprising a nucleic acid, a peptide, a protein, a viral particle, a virus-like particle, a non-viral molecule, a synthetic polymer, and a glycosaminoglycan (GAG)-binding enhanced transduction (GET)-cargo molecule. In one embodiment, the transfection delivery molecule comprises a glycosaminoglycan (GAG)-binding enhanced transduction (GET)-cargo molecule (herein referred to as the “GET system” or “GET molecule”).

The advantageous transfection efficiency of the GET system is described in detail in PCT Publication No: WO2015092417. The use of the GET molecule together with a PKC modifier, and optionally further with HDACi, advantageously generates transfection levels significantly above gold-standard transfection technologies such as Lipofectamine products (from Life Technologies).

In one embodiment, the transfection delivery molecule may be a glycosaminoglycan (GAG)-binding enhanced transduction (GET) delivery molecule comprising:
a cargo
a GAG binding element, which is capable of binding to GAG on the surface of the cell; and
a protein transduction domain.

In another embodiment, the transfection delivery molecule may be a glycosaminoglycan (GAG)-binding enhanced transduction (GET) delivery molecule comprising:
a cargo-binding molecule for binding to a cargo, and optionally wherein the cargo is bound to the cargo-binding molecule;
a GAG binding element, which is capable of binding to GAG on the surface of the cell; and
a protein transduction domain.

The GAG binding element may be different to the protein transduction domain. The GAG binding element may be different in structure and/or sequence to the protein transduction domain. The GAG binding element may be specific to a different cell surface molecule relative to the protein transduction domain. The GAG binding element may be more specific to a cell surface molecule than the protein transduction domain. In one embodiment, the GAG binding element is not a protein transduction domain as described herein. In one embodiment, the protein transduction domain is not a specific GAG-binding element, as described herein. The skilled person will understand that a protein transduction domain may or may not bind GAG on a cell surface, but the binding is not specific or preferential to GAG. The GAG binding element may preferentially bind to GAG relative to non-specific binding of GAG by a protein transduction domain.

The GAG binding element may be a heparin sulphate glycosaminoglycan (HS-GAG) binding element, which is capable of binding to HS-GAG on the surface of the cell.

Heparan sulfate glycosaminoglycan (HS-GAG) is a proteoglycan in which two or three HS chains are attached in close proximity to cell surface or extracellular matrix proteins. It is in this form that HS binds to a variety of protein ligands and regulates a wide variety of biological activities, including developmental processes, angiogenesis, blood coagulation and tumour metastasis. Heparan sulfate is a member of the glycosaminoglycan family of carbohydrates and is very closely related in structure to heparin. Both consist of a variably sulfated repeating disaccharide unit. The most common disaccharide unit within heparan sulfate is composed of a glucuronic acid (GlcA) linked to N-acetylglucosamine (GlcNAc) typically making up around 50% of the total disaccharide units.

The GAG binding element may have specific affinity for GAG. The HS-GAG binding element may have specific affinity for HS-GAG. The HS-GAG binding element may comprise a heparin binding domain (HBD), or a variant thereof. The heparin binding domain variant may comprise a truncated heparin binding domain, or an extended heparin binding domain. The GAG binding element may comprise any protein, peptide or molecule that specifically or preferentially binds to GAG. The HS-GAG binding element may comprise any protein, peptide or molecule that specifically or preferentially binds to HS-GAG.

The HS-GAG binding element may comprise at least part of the heparin binding domain of Heparin-Binding EGF-like Growth Factor (HB-EGF). The heparin binding domain may comprise P21 of HB-EGF. The heparin binding domain may comprise a truncated, extended, or functional variant of P21.

The HS-GAG binding element may comprise a heparin binding domain of a fibroblast growth factor, or a functional variant thereof.

The HS-GAG binding element may be selected from any of the group comprising FGF, antithrombin, such as ATIII, VEGF, BMPs, Wnts, Shh EGFs, and PDGF; or variants thereof. The HS-GAG binding element may comprise any of FGF2, FGF7, or PDGF. The HS-GAG binding element may comprise one or more of the heparin binding sulphate domains of any FGF protein (e.g. domains A, B or C). The HS-GAG binding element may comprise FGF4. The HS-GAG binding element may comprise FGF1 HBD A (heparan sulphate binding domain A (the first HBD domain of FGF1)), FGF2 HBD A (heparan sulphate binding domain A), FGF4 HBD A (heparan sulphate binding domain A), FGF1 HBD C (heparan sulphate binding domain C), FGF2 HBD B (heparan sulphate binding domain B), FGF2 HBD C (heparan sulphate binding domain C), FGF4 HBD C (heparan sulphate binding domain C), FGF7 HBD B (heparan sulphate binding domain B), FGF7 HBD C (heparan sulphate binding domain C), antithrombin, such as ATIII, VEGF, or PDGF, or variants thereof.

The HS-GAG binding element may be selected from any of the group comprising Hepatocyte Growth Factor, Interleukin, morphogens, HS-GAG binding enzymes, Wnt/Wingless, Endostatin, viral protein, such as foot and mouth disease virus protein, annexin V, lipoprotein lipase; or HS-GAG binding fragments thereof. The HS-GAG binding element may comprise any protein, peptide or molecule capable of specifically binding HS-GAG.

A “variant” may be understood by the skilled person to include a functional variant, wherein there may be some sequence differences from the known, reported, disclosed or claimed sequence, but the variant may still bind to HS-GAG. Conservative amino acid substitutions are also envisaged within the meaning of “variant”.

The HS-GAG binding element may comprise the amino acid sequence KRKKKGKGLGKKRDPCLRKYK (P21, SEQ ID NO. 1). The HS-GAG binding element may comprise a sequence having at least 80% identity to SEQ ID NO. 1. The HS-GAG binding element may comprise a sequence having at least 90% identity to SEQ ID NO. 1. The HS-GAG binding element may comprise a sequence having at least 95% identity to SEQ ID NO. 1. The HS-GAG binding element may comprise a sequence having at least 98% identity to SEQ ID NO. 1. The HS-GAG binding element may comprise a sequence having at least 99% identity to SEQ ID NO. 1.

The HS-GAG binding element may comprise the amino acid sequence GRP RE S G K K R K R K R L K P T (PDGF, SEQ ID NO. 3). The HS-GAG binding element may comprise a sequence having at least 80% identity to SEQ ID NO. 3. The HS-GAG binding element may comprise a sequence having at least 90% identity to SEQ ID NO. 3. The HS-GAG binding element may comprise a sequence having at least 95% identity to SEQ ID NO. 3. The HS-GAG binding element may comprise a sequence having at least 98% identity to SEQ ID NO. 3. The HS-GAG binding element may comprise a sequence having at least 99% identity to SEQ ID NO. 3.

The HS-GAG binding element may comprise the amino acid sequence T Y A S A K W T H N G G E M F V A L N Q ((FGF7, HBD B) SEQ ID NO. 5). The HS-GAG binding element may comprise a sequence having at least 80% identity to SEQ ID NO. 5. The HS-GAG binding element may comprise a sequence having at least 90% identity to SEQ ID NO. 5. The HS-GAG binding element may comprise a sequence having at least 95% identity to SEQ ID NO. 5. The HS-GAG binding element may comprise a sequence having at least 98% identity to SEQ ID NO. 5. The HS-GAG binding element may comprise a sequence having at least 99% identity to SEQ ID NO. 5.

The HS-GAG binding element may comprise the amino acid sequence T Y R S R K Y T S W Y V A L K R ((FGF2, HBD B) SEQ ID NO. 7). The HS-GAG binding element may comprise a sequence having at least 80% identity to SEQ ID NO. 7. The HS-GAG binding element may comprise a sequence having at least 90% identity to SEQ ID NO. 7. The HS-GAG binding element may comprise a sequence having at least 95% identity to SEQ ID NO. 7. The HS-GAG binding element may comprise a sequence having at least 98% identity to SEQ ID NO. 7. The HS-GAG binding element may comprise a sequence having at least 99% identity to SEQ ID NO. 7.

Sequence identity may be determined by standard BLAST alignment parameters (provided by http://www.ncbi.nlm.nih.gov/).

The GAG binding element may comprise a GAG binding antibody, or a variant or fragment thereof. The HS-GAG binding element may comprise a HS-GAG binding antibody, or a variant or fragment thereof. The antibody fragment may be an antibody variable domain, an scFv, a diabody, a FAb, a Dab, a F(ab)′2, a heavy-light chain dimer, or a single chain structure. The antibody variant may comprise a protein scaffold comprising CDRs, an antibody mimetic, or a DARPin.

The GAG or HS-GAG binding element may comprise a nanobody (single-domain antigen-binding fragments derived from heavy-chain antibodies that are devoid of light chains and occur naturally in Camelidae).

The single-domain antibody may comprise a V_{H}H fragment comprising a CDR1, CDR2 and CDR3 wherein
CDR1 may comprise or consist of the amino acid sequence of GFTVSSNE or GFAF SSYA;
CDR2 may comprise or consist of the amino acid sequence of ISGGST or IGTGGDT; and
CDR3 may comprise or consist of the amino acid sequence of GRRLKD or SLRMNGWRAHQ.

The single-domain antibody may comprise a V_{H}H fragment comprising a CDR1, CDR2 and CDR3 wherein
CDR1 may comprise or consist of the amino acid sequence of GFTVSSNE;
CDR2 may comprise or consist of the amino acid sequence of ISGGST; and
CDR3 may comprise or consist of the amino acid sequence of GRRLKD.

Alternatively, the CDR3 may comprise the amino acid sequence GMRPRL, HAPLRNTRTNT, GSRSSR, GRTVGRN, GKVKLPN, SGRKGRMR, SLRMNGWRAHQ, or RRYALDY.

The single-domain antibody may comprise a V_{H}H fragment comprising a CDR1, CDR2 and CDR3 wherein
CDR1 may comprise or consist of the amino acid sequence of GFAFSSYA;
CDR2 may comprise or consist of the amino acid sequence of IGTGGDT; and
CDR3 may comprise or consist of the amino acid sequence of SLRMNGWRAHQ.

Alternatively, the CDR3 may comprise the amino acid sequence LKQQGIS, AMTQKKPRKLSL, HAPLRNTRTNT, GMRPRL, RRYALDY, or SGRKYFRARDMN.

The HS-GAG binding element may comprise anti-HS scFv antibodies AO4B08, AO4B05, AO4F12, RB4CB9, RB4CD12, RB4EA12, or RB4EG12 (as described in Jenniskens et al (2000. The Journal of Neuroscience, 20(11):4099-4111) and Smits, et al (2006. METHODS IN ENZYMOLOGY, VOL. 416, pp. 61-87) incorporated herein by reference); or fragments thereof. The HS-GAG binding element may comprise AO4B08. The HS-GAG binding element may comprise CDR1, CDR2 and CDR3 of AO4B08, AO4B05, AO4F12, RB4CB9, RB4CD12, RB4EA12, or RB4EG12. The HS-GAG binding element may comprise CDR1, CDR2 and CDR3 of AO4B08.

The HS-GAG binding element may comprise HS3A8, LKIV69, EW3D10, EW4G2, NS4F5, RB4EA12, HS4E4 or HS4C3 (as described in Wijnhoven et al (2008) Glycoconj J 25:177-185) and Smits, et al (2006. METHODS IN ENZYMOLOGY, VOL. 416, pp. 61-87) incorporated herein by reference). The HS-GAG binding element may comprise HS4E4 or HS4C3. The HS-GAG binding element may comprise CDR1, CDR2 and CDR3 of HS3A8, LKIV69, EW3D10, EW4G2, NS4F5, RB4EA12, HS4E4 or HS4C3. The HS-GAG binding element may comprise CDR1, CDR2 and CDR3 of HS4E4 or HS4C3.

The HS-GAG binding element may comprise SEQ ID NO: 15 or 17 (A04B08). The HS-GAG binding element may comprise SEQ ID NO: 11 or 13 (HS4C3). The HS-GAG binding element may comprise an antibody, or antibody fragment, heavy chain and/or light chain. The HS-GAG binding element may comprise an antibody, or antibody fragment, heavy chain, comprising HCDR1, HCDR2 and HCDR3 chains and/or light chain, comprising LCDR1, LCDR2 and LCDR3.

In one embodiment of the invention, the protein transduction domain is not GAG. In another embodiment, the protein transduction domain is different to GAG.

The protein transduction domain may be hydrophilic or amphiphilic. The protein transduction domain may comprise a majority of hydrophilic amino acid residues. The protein transduction domain may comprise a majority of arginine and/or lysine amino acid residues. The protein transduction domain may comprise a periodic sequence, having a repeated amino acid sequence motif. The protein transduction domain may comprise penetratin, TAT such as HIV derived TAT, MAP, or transportan, pVec, or pep-1.

Where reference is made to a “majority” of residue, this may be understood by the skilled person to include greater than 50% of the residues. A majority may be 55%, 60%, 70%, 80%, 90% or 95% of the residues.

The protein transduction domain may be selected from any of the group comprising:
Penetratin or Antenapedia PTD RQIKWFQNRRMKWKK;
HIV transactivator protein (TAT) YGRKKRRQRRR;
Synembryn B (SynB)1 RGGRLSYSRRRFSTSTGR;
SynB3 RRLSYSRRRF;
PTD-4 PIRRRKKLRRLK;
PTD-5 RRQRRTSKLMKR;
Flock house virus (FHV) Coat-(35-49) RRRRNRTRRNRRRVR;
Brome mosaic virus (BMV) Gag-(7-25) KMTRAQRRAAARRNRWTAR;
Human T-cell lymphotrophic virus (HTLV)-II Rex-(4-16) TRRQRTRRARRNR;
D-Tat GRKKRRQRRRPPQ;
R9-Tat GRRRRRRRRRPPQ;
Transportan GWTLNSAGYLLGKINLKALAALAKKIL chimera;
Microtubule-associated protein (MAP) KLALKLALKLALALKLA;
Streptavidin-binding peptide (SBP) MGLGLHLLVLAAALQGAWSQPKKKRKV;
Folate-binding protein (FBP) GALFLGWLGAAGSTMGAWSQPKKKRKV;
Human 3-methyladenine-DNA glycosylase) (MPG) ac-GALFLGFLGAAGSTMGAWSQPKKKRKV-cya;
10 MPG-nuclear localisation sequence (NLS) ac-GALFLGFLGAAGSTMGAWSQPKSKRKV-cya;
Pep-1 ac-KETWWETWWTEWSQPKKKRKV-cya; and
Pep-2 ac-KETWFETWFTEWSQPKKKRKV-cya; or
polyarginines, such as R×N (4<N<17) chimera, polylysines, such as K×N (4<N<17) chimera, (RAca)6R, (RAbu)6R, (RG)6R, (RM)6R, (RT)6R. (RS)6R, R10, (RA)6R, R7, and R8.

The protein transduction domain may comprise polyarginine or polylysine. The protein transduction domain may comprise an arginine and lysine repeat sequence. The protein transduction domain may comprise arginine residues, such as consecutive arginine residues. The protein transduction domain may consist essentially of arginine residues. The protein transduction domain may comprise arginine repeats, such as 4-20 arginine residues. The protein transduction domain may comprise 8 arginine residues. The protein transduction domain may comprise between about 6 and about 12 arginine residues. The protein transduction domain may comprise between about 7 and about 9 arginine residues.

The protein transduction domain may comprise between about 4 and about 12 amino acid residues. The protein transduction domain may comprise between about 6 and about 12 amino acid residues. The protein transduction domain may comprise between about 7 and about 9 amino acid residues. The protein transduction domain may comprise at least about 4 amino acid residues. The protein transduction domain may comprise at least about 6 amino acid residues.

The protein transduction domain may comprise lysine residues, such as consecutive lysine residues. The protein transduction domain may consist essentially of lysine residues. The protein transduction domain may comprise lysine repeats, such as 4-20 lysine residues. The protein transduction domain may comprise 8 lysine residues. The protein transduction domain may comprise between about 4 and about 12 lysine residues. The protein transduction domain may comprise between about 6 and about 12 lysine residues. The protein transduction domain may comprise between about 7 and about 9 lysine residues.

The protein transduction domain may comprise Q and R residues, such as consecutive QR repeat residues. The protein transduction domain may consist essentially of Q and R residues. The protein transduction domain may comprise QR repeats, such as 4-20 QR repeat residues. The protein transduction domain may comprise 8 QR repeat residues. The protein transduction domain may comprise between about 6 and about 12 QR repeat residues. The protein transduction domain may comprise between about 7 and about 9 QR repeat residues.

In one embodiment the cargo is bound to the cargo-binding molecule. The cargo may be bound to the cargo-binding molecule during manufacture of the delivery molecule, post-manufacture, prior to use, or during use.

The cargo-binding molecule may be a carrier for the cargo molecule. A single cargo-binding molecule may bind and carry multiple cargo molecules. The cargo-binding molecule may protect the cargo prior to internalisation into a cell. The cargo-binding molecule may be capable of binding to biotin on a biotinylated cargo. The cargo binding molecule may be capable of binding to nucleic acid-based cargo. The cargo-binding molecule may be capable of binding to a peptide-based cargo. The cargo-binding molecule may be capable of binding to an antibody cargo, or fragment or mimetic thereof. The cargo-binding molecule may be capable of binding to a nanoparticle cargo, such as a metal or polymer nanoparticle. The cargo-binding molecule may be functionally inactive in a cell, but can carry or bind to an active cargo. The cargo-binding molecule may comprise a chemical linker molecule. The cargo-binding molecule may comprise an affinity tag. The cargo-binding molecule may comprise a peptide or protein. The cargo-binding molecule may comprise mSA2 (monomeric streptavidin 2). The cargo-binding molecule may comprise a nucleic acid interacting peptide, such as LK15. The cargo-binding molecule may comprise an antibody binding molecule, such as an IgG binding protein. The IgG binding protein may comprise S. aureus IgG ninding protein SpAB. The skilled person will understand that any suitable pairs or groups of molecules may be used for the cargo and cargo-binding molecule provided that they have sufficient binding or affinity between them.

The bond or interaction between the cargo and the cargo-binding molecule may be reversible, or degradeable, for example in the intracellular environment.

The GAG binding element and protein transduction domain may be bound to the cargo and/or cargo-binding molecule by direct chemical conjugation or through a linker molecule. The GAG binding element and protein transduction domain may be bound to the cargo by direct chemical conjugation or through a linker molecule. The GAG binding element and protein transduction domain may be bound to the cargo-binding molecule by direct chemical conjugation or through a linker molecule. The GAG binding element, protein transduction domain and cargo may be a single fusion molecule (e.g. it may be encoded and transcribed as a single peptide molecule). The GAG binding element, protein transduction domain and cargo-binding molecule may be a single fusion molecule (e.g. it may be encoded and transcribed as a single peptide molecule). The protein transduction domain and GAG binding element may flank the cargo-binding molecule and/or cargo.

The delivery molecule may be between about 10 and about 30,000 amino acids in length. The delivery molecule may be between about 20 and about 30,000 amino acids in length. The delivery molecule may be between about 30 and about 30,000 amino acids in length. The delivery molecule may be between about 40 and about 30,000 amino acids in length. The delivery molecule may be between about 10 and about 10,000 amino acids in length. The delivery molecule may be between about 20 and about 10,000 amino acids in length. The delivery molecule may be between about 40 and about 10,000 amino acids in length. The delivery molecule may be between about 10 and about 3,000 amino acids in length. The delivery molecule may be between about 20 and about 3,000 amino acids in length. The delivery molecule may be between about 40 and about 3,000 amino acids in length. The delivery molecule may be between about 10 and about 1000 amino acids in length. The delivery molecule may be between about 20 and about 1000 amino acids in length. The delivery molecule may be between about 40 and about 1000 amino acids in length. The delivery molecule may be between about 40 and about 500 amino acids in length. The delivery molecule may be between about 10 and about 500 amino acids in length. The delivery molecule may be between about 20 and about 500 amino acids in length. The delivery molecule may be between about 100 and about 3,000 amino acids in length. The delivery molecule may be at least about 100 amino acids in length.

The delivery molecule may be a single fusion molecule. The cargo, HS-GAG binding element, and protein transduction domain may be fused together. The HS-GAG binding element and protein transduction domain may flank the cargo. The cargo, HS-GAG binding element, and protein transduction domain may be linked together by one or more linker molecules.

The delivery molecule may have a molecular weight of at least 1 KDa. The delivery molecule may have a molecular weight of at least 5 KDa. The delivery molecule may have a molecular weight of at least 10 KDa. The delivery molecule may have a molecular weight of at least 20 KDa. The delivery molecule may have a molecular weight of 400 KDa or less. The delivery molecule may have a molecular weight of 300 KDa or less. The delivery molecule may have a molecular weight of between about 0.5 KDa and about 400 kDa. The delivery molecule may have a molecular weight of between about 1 KDa and about 400 kDa. The delivery molecule may have a molecular weight of between about 0.5 KDa and about 200 kDa. The delivery molecule may have a molecular weight of between about 1 KDa and about 200 kDa. The delivery molecule may have a molecular weight of between about 2 KDa and about 300 kDa. The delivery molecule may have a molecular weight of between about 20 KDa and about 300 kDa. The delivery molecule may have a molecular weight of between about 20 KDa and about 100 kDa.

The cargo may be capable of binding, such as ionic or covalent binding, to the protein transduction domain and/or GAG binding element.

In one embodiment, the transfection delivery molecule comprises the molecule for delivery (the cargo). In particular, the molecule for delivery (i.e. the cargo) and the transfection delivery molecule may be termed the “transfection delivery molecule”.

### The Cell/Population of Cells

The cell may be a mammalian cell, such as a human cell. The cell may be a cancerous cell. The cell may be a stem cell. The cell may be a mutant cell. The cell may comprise a population of cells. The population of cells may be a mixed population of cell types. The cell may be a mesenchymal stem cell, such as iHMSCs. The cell may be an embryonic stem cell. The cell may be a pluripotent stem cell, such as a human pluripotent stem cell (hPSC).

In one embodiment, the cell or population of cells for transfection is a blood cell, such as LCL or RAW246.7. In one embodiment, the cell or population of cells for transfection is a microglia, such as BV2.

In one embodiment, the cell or population of cells for transfection may be selected from the group comprising peripheral blood mononuclear cells (PBMCs), hematopoietic stem cells (HSCs), mesenchymal stem cells (MSCs), induced pluripotent stem cells (IPSCs), human embryonic stem cells (HESCs), sperm, oocytes, skeletal muscle cells, brain cells, and lung cells, or combinations thereof.

The lung cells may comprise alveolar cells. The brain cells may comprise neurons and/or glial cells. The skeletal muscle cells may comprise myocytes and/or myoblasts.

The skilled person will recognise that the number of cells to be transfected may vary depending on the application, and whether the transfection is in vivo or in vitro. In one embodiment, for example in vitro, the number of cells to be transfected comprises at least 1000 cells, 5000 cells, 1×10³ cells, 5×10³ cells, or 8×10³ cells, or more. In another embodiment, for example in vivo, the number of cells to be transfected comprises at least 1000 cells, 5000 cells, 1×10³ cells, 5×10³ cells, 8×10³ cells, 1×10⁴ cells, 1×10⁵ cells, 1×10⁶ cells, 1×10⁷ cells, or 1×10⁸ cells, or more.

The method may further comprise centrifugation of the delivery molecule with the cells to be transfected, for example to facilitate their contact and enhance transfection efficiency.

### Other Method Details

The method may be carried out in vitro or in vivo. In an embodiment wherein the method is carried out in vivo, the cells to be transfected may be within a subject. The subject may be mammalian, such as human.

In an embodiment wherein the method is carried out in vitro, the cells to be transfected may be isolated from a subject. The cells may be a maintained cell line, or maintained cell culture extracted from a subject.

In an embodiment wherein the method is carried out in vivo, the PKC modulator and/or the molecule for transfection may be administered to the subject. In an embodiment further providing HDAC inhibition, the HDAC inhibitor may be administered to the subject. The PKC modulator and/or the molecule for transfection may be provided in a pharmaceutically acceptable excipient. The HDAC inhibitor may be provided in a pharmaceutically acceptable excipient. The administration may be systemic and/or directly upon the in situ cells to be transfected.

The method of transfecting a cell with a molecule may be a non-viral method of transfection. A “non-viral method of transfection” may be understood by the skilled person to encompass any method of transfection, wherein the molecule for transfection is not viral in origin.

Advantageously, non-viral methods of transfection allow for a shorter duration of transgene expression, enable a flexible size of DNA to be transported, are less expensive, easier to prepare, and generate little or no in vivo immune response, compared to viral methods of transfection.

An in vitro transfection may be carried out in cell growth media, buffer or saline.

### pH Mediated Endosomal Escape

In one embodiment, the transfection delivery molecule may be used, such as delivered, in combination with a pH responsive peptide comprising between about 5 and 20 histidine residues.

Histidine exhibits considerable buffer capacity, so would be protonated at the low pH values of the late endosome or lysosomes. There is a “proton-sponge” hypothesis describing that unprotonated residues such as the imidazole ring of Histidine can absorb protons as they are pumped into the endosome/lysosome, resulting in more protons being pumped in, leading to an increased influx of Cl⁻ ions and water. A combination of the osmotic swelling and a swelling of positively charged residues in cargo because of repulsion between protonated amine groups, causes the rupture of the endo/lysosomal membrane with subsequent release of its contents into the cytoplasm. The increased proton transport into late endosomes and lysosomes is by ATP driven V-ATPase. This pump is capable of continuous transport of protons and as long as there is sufficient ATP available in the cytosol the V-ATPase activity aims to retain the proton gradient across the vesicle membrane. The GET peptides are likely to buffer the lysosome, however, the V-ATPase pump keeps the bulk of the vesicle acidic by increasing the influx of protons. No change in pH is observed, even with the increased influx of protons, because of the “proton sponge” hypothesis.

In one embodiment the pH responsive peptide comprising between about 5 and 20 histidine residues may be added to the transfection delivery molecule, such as the GAG peptide described herein. In particular, the pH responsive peptide may be bound to, or complexed with, the transfection delivery molecule, such as the GAG peptide described herein. The pH responsive peptide may be covalently bound to the transfection delivery molecule, such as the GAG peptide described herein, for example, in the form of a fusion peptide.

In another embodiment, the pH responsive peptide may be an accessory peptide that is provided along with the transfection delivery molecule. In one embodiment the pH responsive peptide may be contained within a nanoparticle which is released in endosomal vesicles.

In another embodiment, the pH responsive peptide may replace the PTD (protein transduction domain/CPP) of the transfection delivery molecule, such as the GAG peptide described herein.

In one embodiment, the pH responsive peptide comprises or consists of 5-20 histidine residues and a nucleic acid-interacting sequence. In another embodiment, the pH responsive peptide comprises or consists of 5-12 histidine residues and a nucleic acid-interacting sequence. In another embodiment, the pH responsive peptide comprises or consists of 8-20 histidine residues and a nucleic acid-interacting sequence. In another embodiment, the pH responsive peptide comprises or consists of 5-15 histidine residues and a nucleic acid-interacting sequence. In another embodiment, the pH responsive peptide comprises or consists of 8-15 histidine residues and a nucleic acid-interacting sequence. In another embodiment, the pH responsive peptide comprises or consists of 8-12 histidine residues and a nucleic acid-interacting sequence. In another embodiment, the pH responsive peptide comprises or consists of 9-11 histidine residues and a nucleic acid-interacting sequence. In another embodiment, the pH responsive peptide comprises or consists of 10 histidine residues and a nucleic acid-interacting sequence.

The nucleic acid-interacting sequence may be capable of biding nucleic acid, preferably non-specifically, for example via its charge. The nucleic acid-interacting sequence may be amphipathic, thereby also having endosomal escape function. The nucleic acid-interacting sequence may be between 5 and 30 residues in length. The nucleic acid-interacting sequence may be no more than 25, 30 or 40 residues in length. The nucleic acid-interacting sequence may comprise or consist of a plurality of K and/or L residues. The nucleic acid-interacting sequence may comprise or consist of 10-25 K and/or L residues. The nucleic acid-interacting sequence may comprise or consist of 15 K and/or L residues. The K and L residues may alternate, or may the pH responsive peptide may comprise repeating units of KLL and/or KLLL. The nucleic acid-interacting sequence may comprise or consist of KLLKLLLKLLLKLLK (SEQ ID NO: 19), or a variant thereof. The variant may have at least 80%, 85%, 90%, 95%, 98% or 99% identity with SEQ ID NO: 19.

In one embodiment, the pH responsive peptide comprises or consists of the sequence KLLKLLLKLLLKLLKHHHHHHHHHH (termed “LK15-10H” herein) (SEQ ID NO: 20), or a variant thereof. The variant may have at least 80%, 85%, 90%, 95%, 98% or 99% identity with SEQ ID NO: 20. In one embodiment, the pH responsive peptide comprises or consists of the sequence KLLKLLLKLLLKLLK(H₅₋₂₀) (SEQ ID NO: 21).

In an embodiment wherein the pH responsive peptide is bound to the transfection delivery molecule, such as the GAG peptide, the pH responsive peptide may replace the protein transduction domain of the transfection delivery molecule, such as the GAG peptide. In another embodiment wherein the pH responsive peptide is bound to the transfection delivery molecule, such as the GAG peptide, the pH responsive peptide may be provided in addition to the protein transduction domain of the transfection delivery molecule, such as the GAG peptide.

In one embodiment, the transfection delivery molecule with a pH responsive peptide comprises or consists of the sequence: FLHTYRSRKYTSWYVALKRKLLKLLLKLLLKLLKHHHHHHHHHH (SEQ ID NO: 22) (FGF2B-LK15-10H) (also termed “44” herein).

In one embodiment, the transfection delivery molecule with a pH responsive peptide comprises or consists of the sequence: TYRSRKYTSWYVALKRKLLKLLLKLLLKLLKHHHHHHHHHHRRRRRRRR (SEQ ID NO: 23) (FGF2B-LK15-10H-8R) (also termed “FLHR” herein).

In one embodiment, the transfection delivery molecule with a pH responsive peptide comprises or consists of the sequence: TYRSRKYTSWYVALKRKLLKLLLKLLLKLLKRRRRRRRRHHHHHHHHHH (SEQ ID NO: 24) (FGF2B-LK15-8R-10H) (also termed “FLRH” herein).

In one embodiment, the transfection delivery molecule with a pH responsive peptide comprises or consists of the sequence: TYRSRKYTSWYVALKRRRRRRRRRKLLKLLLKLLLKLLKHHHHHHHHHH (SEQ ID NO: 25) (FGF2B-8R-LK15-10H) (also termed “FRLH” herein)

### Other Aspects

According to another aspect of the invention, there is provided a composition comprising:
a modulator of PKC and/or a pH responsive peptide comprising between about 5 and 20 histidine residues; and
a molecule for transfection.

Preferably, the 5-20 histidine residues of the pH responsive peptide are capable of being protonated in an acidic environment of an endosome.

In one embodiment of the composition, the modulator of PKC may be a PKC inhibitor. The composition may further comprise a HDAC inhibitor. The composition may be a pharmaceutically acceptable composition.

According to another aspect of the invention, there is provided a kit comprising:
a modulator of PKC and/or a pH responsive peptide comprising between about 5 and 20 histidine residues; and
a molecule for transfection.

In one embodiment of the kit, the modulator of PKC may be a PKC inhibitor.

The kit may further comprise a HDAC inhibitor. The components of the kit may be provided separately or as a composition.

The kit may further comprise a centrifuge tube, and optionally a centrifuge.

The molecule for transfection may be in lyophilised form. The kit may additionally comprise a reconstitution solution, such as a buffer.

According to another aspect of the invention, there is provided a method of enhancing transfection of a cell with a molecule for transfection, the method comprising the steps of:
adding the molecule for transfection to the cells; and
modulating the activity of protein kinase C (PKC) in the cell and/or providing a pH responsive peptide comprising between about 5 and 20 histidine residues to enhance endosomal release in the cell.

The pH responsive peptide may be protonated in the endosome post internalisation.

The molecule for transfection may not be in itself capable of modulating the activity of PKC in the cell. The molecule for transfection may not comprise or consist of a PKC modulator, such as an inhibitor or activator.

According to another aspect of the present invention, there is provided the use of a modulator of PKC and/or a pH responsive peptide comprising between about 5 and 20 histidine residues for increasing cell transfection efficiency.

Optionally, a HDAC modulator, in combination with the modulator of PKC may be used for increasing cell transfection efficiency. In particular, the use may be in combination with a HDAC inhibitor. The use may be with GET-mediated transfection.

The method of the invention may be used in gene therapy.

According to another aspect of the invention, there is provided a method of gene therapy comprising administering to a subject:

(a) a modulator of PKC and/or providing a pH responsive peptide comprising between about 5 and 20 histidine residues; and
(b) a molecule for transfection, wherein the molecule for transfection comprises nucleic acid.

The method of gene therapy may further comprise the administration of a HDAC inhibitor.

According to another aspect of the invention, there is provided a method of treatment of a subject comprising administering to the subject:

(a) a modulator of PKC and/or providing a pH responsive peptide comprising between about 5 and 20 histidine residues; and
(b) a molecule for transfection, wherein the molecule for transfection comprises a therapeutically effective molecule.

The therapeutically effective molecule may be a nucleic acid, protein or peptide as described herein. The therapeutically effective molecule may be a nucleic acid encoding a gene sequence and/or a regulatory sequence, for example as described herein.

According to another aspect of the invention, there is provided a method of treatment of a subject comprising administering to the subject cells that have been transformed according the method of the invention herein.

The cells that have been transformed according the method of the invention herein may intracellularly comprise the molecule for transfection.

According to another aspect of the invention, there is provided a modulator of PKC and/or providing a pH responsive peptide comprising between about 5 and 20 histidine residues for use in combination with a molecule for transfection as a medicament.

The use may be for gene therapy. In another embodiment the use may be for treatment or prevention of a disease. In another embodiment the use may be for tissue repair or replacement. The tissue may be soft tissue or bone tissue.

The treatment or prevention may be for treatment or prevention of a monogenic disease. The monogenic disease may be selected from any of the group comprising sickle cell disease, cystic fibrosis, polycystic kidney disease, Tay-Sachs disease; al-antitrypsin deficiency; and primary ciliary dyskinesia.

The treatment or prevention may be for treatment or prevention of a disease or condition that can be treated or prevented by growth factor overexpression. The treatment or prevention may be for treatment or prevention of a disease or condition comprising wound healing, tissue repair, bone repair, diabetic ulcers, neurodegenerations (e.g. Alzheimers or Parkinsons), or osteoarthritis.

### Definitions

A “modulator” may be understood by the skilled person to describe an agonist, inverse agonist, or antagonist, i.e. the modulator may increase or decrease the activity of its target.

An “inhibitor” may be understood by the skilled person to describe an inverse agonist or antagonist, i.e. the inhibitor reduces or stops the activity of its target.

The term “temporary modulation”, including “temporary inhibition” or “temporary activation” is understood to be a modulation of an activity in the cell that is not permanent. For example, upon withdrawal or absence of a modulating agent, the original cell activity may be restored. In contrast a permanent modulation may comprise a genetic modulation, such as a knockout of a gene by mutation.

The terms “transfection” and “transduction” maybe used interchangeably herein. They are understood to mean the process of introducing a molecule, such as nucleic acid or protein, into a cell.

The terms “transfected”, “transduced”, or “transformed” in relation to a cell is understood to mean that a cell has internalised a molecule for transfection. The molecule for transfection may be in the cytoplasm and/or the nucleus i.e. not be sequestered in an endosome. Successful transfection may be measured via reporter gene expression (such as GFP or other fluorescent proteins and flow cytometry). The skilled person will understand that the method may not require the determination of transfection rate or efficiency each time or for practice of the method. The term “gene therapy” is understood to be the introduction of genetic material, such as genes and/or regulatory elements, into a cell or cells of a subject to provide a therapeutic benefit or prevent a condition or disease.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

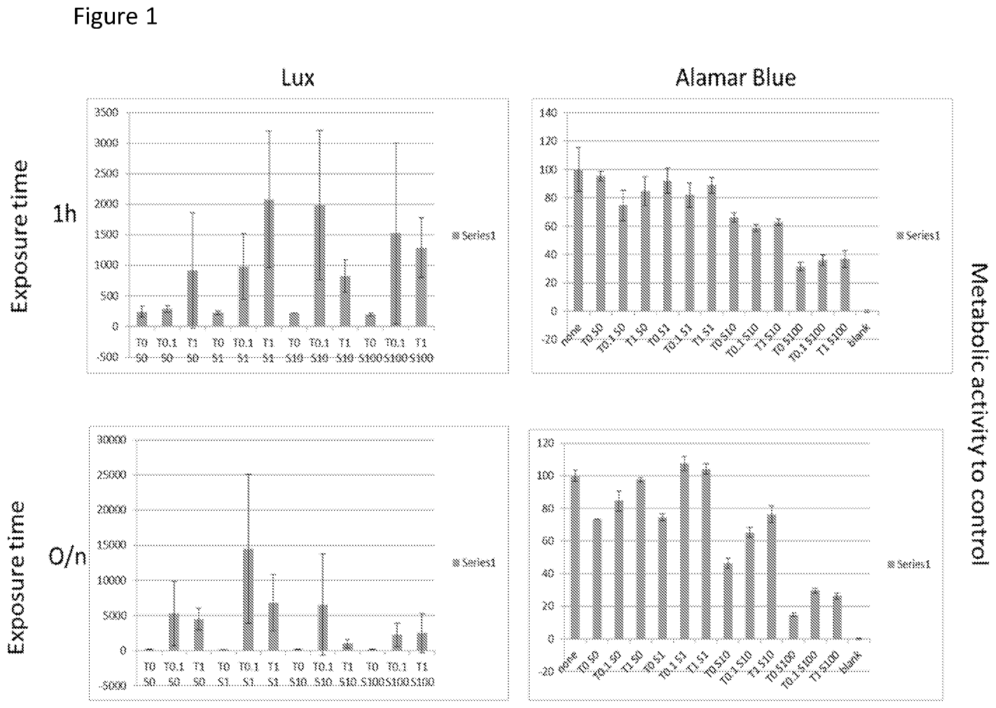

FIG. 1—TPA (T) significantly enhances transfection of T2 cells. Experiment on 20,000 T2 hybrid human T/B cells, measured at 24 hours post-transfection of 1 μg pCMV-Gluc pDNA with GET. T=TPA(μM), S=SAHA (μM). TPA is shown as the major effect on transfection efficiency in T2 cells.

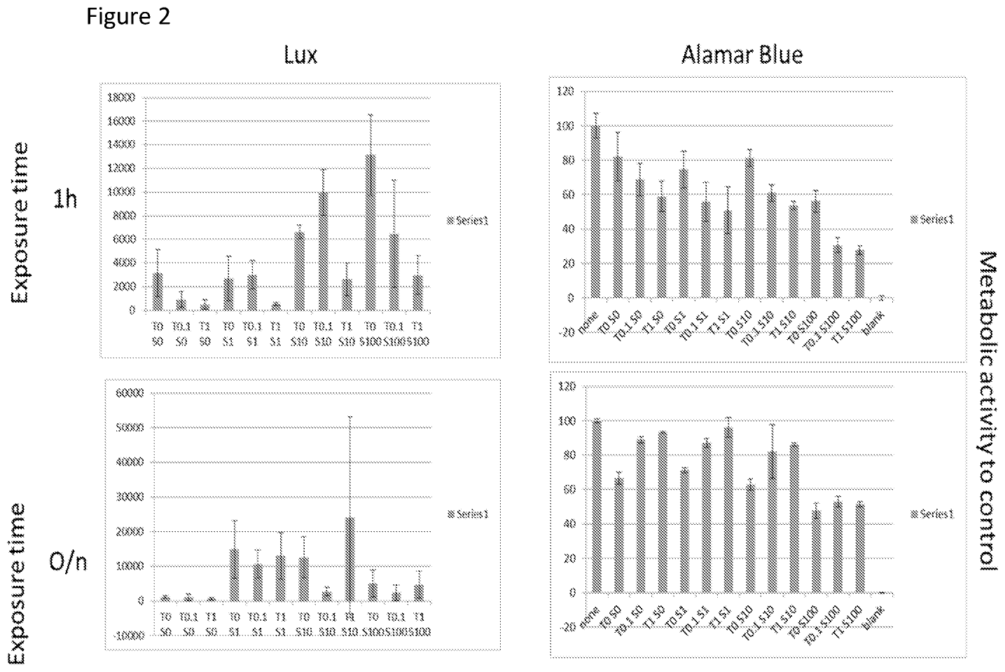

FIG. 2—SAHA (S) significantly enhances transfection of RAW264.7 cells. Experiment on 20,000 RAW264.7 mouse macrophage cells, measured at 24 hours post-transfection of 1 μg pCMV-Gluc pDNA with GET. T=TPA(μM), S=SAHA (μM).

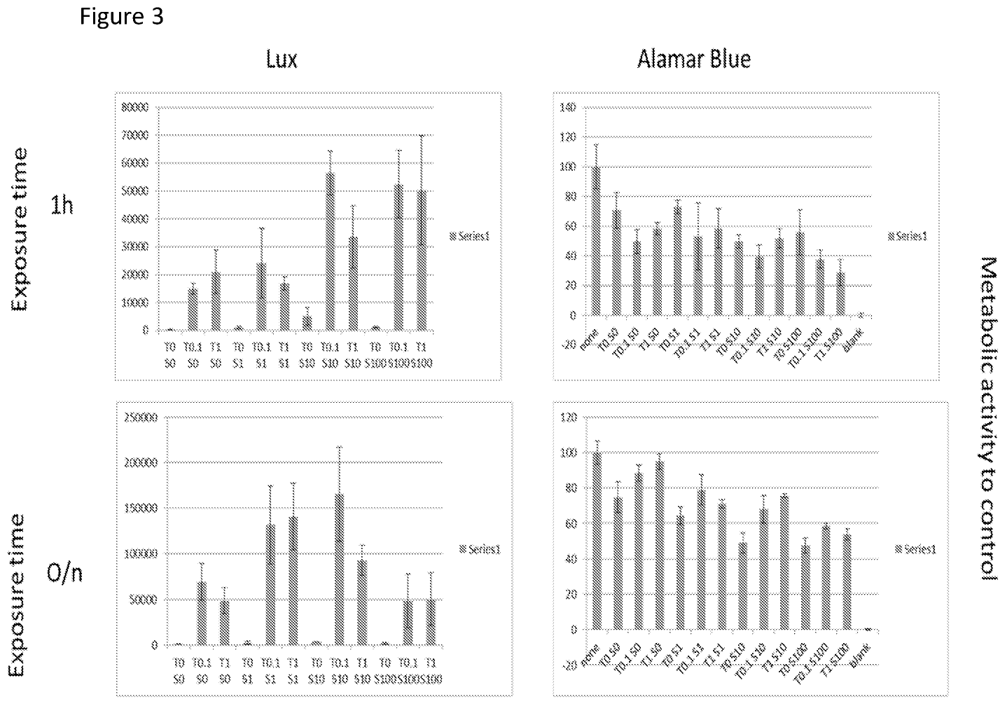

FIG. 3—SAHA (S) and short exposures of TPA (T) significantly enhances transfection of LCL cells. Experiment on 20,000 LCL lymphoblastoid human B cells, measured at 24 hours post-transfection of 1 μs pCMV-Gluc pDNA with GET. T=TPA(μM), S=SAHA (μM). TPA is shown to have the most effect on transfection in LCL cells, and it is enhanced by SAHA. There is effectively no transfection without these modulators.

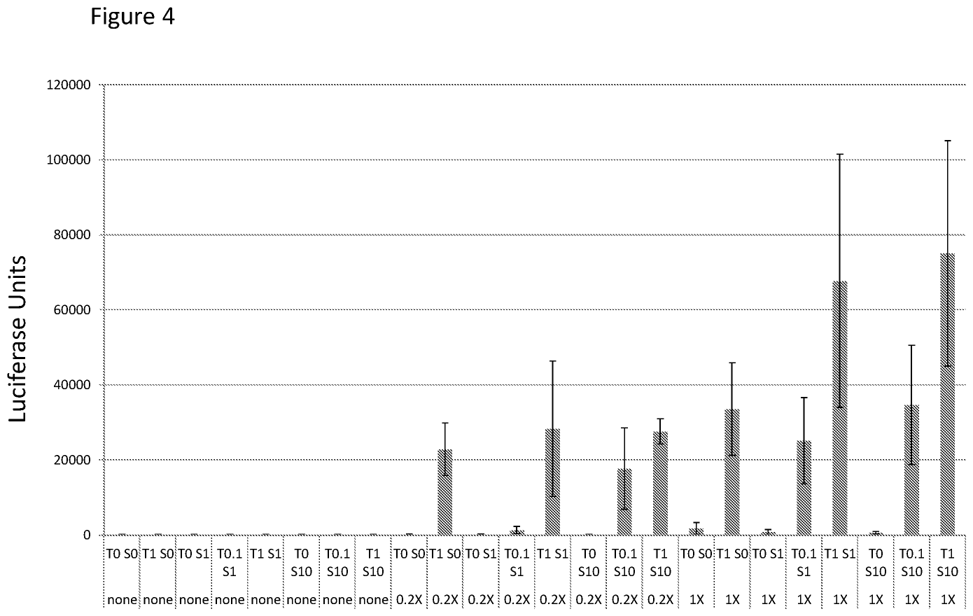

FIG. 4—Transfection of T2 cells. Experiment on 20,000 T2 hybrid human T/B cells, measured at 24 hours post-transfection of pCMV-Gluc pDNA with GET. 1×=1 μg pCMV-Gluc pDNA with GET. 0.2×=0.2 μg pCMV-Gluc pDNA with GET. T=TPA(μM), S=SAHA (μM).

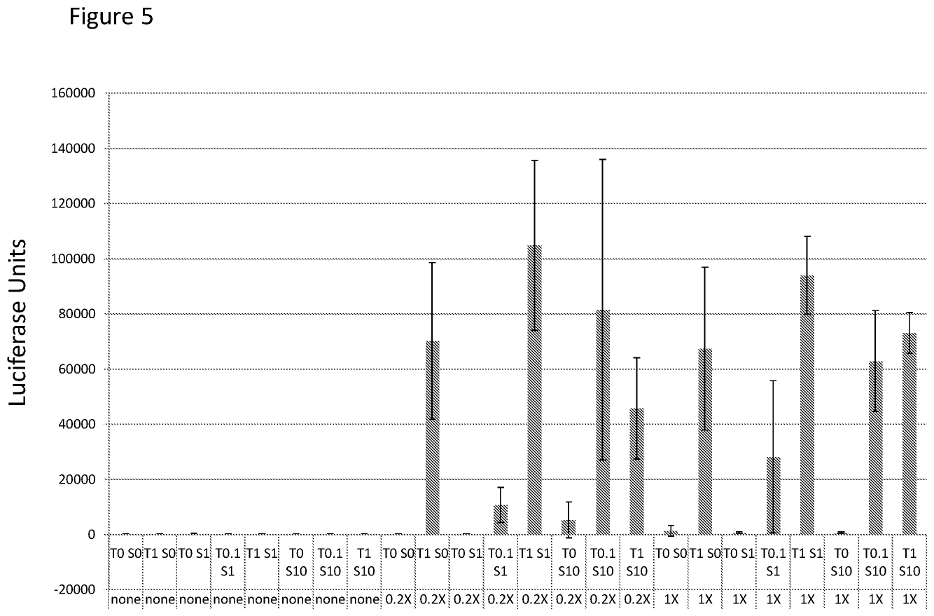

FIG. 5—Transfection of LCL cells. Experiment on 20,000 LCL lymphoblastoid human B cells, measured at 24 hours post-transfection of pCMV-Gluc pDNA with GET. 1×=1 μg pCMV-Gluc pDNA with GET. 0.2×=0.2 μg pCMV-Gluc pDNA with GET. T=TPA(μM), S=SAHA (μM).

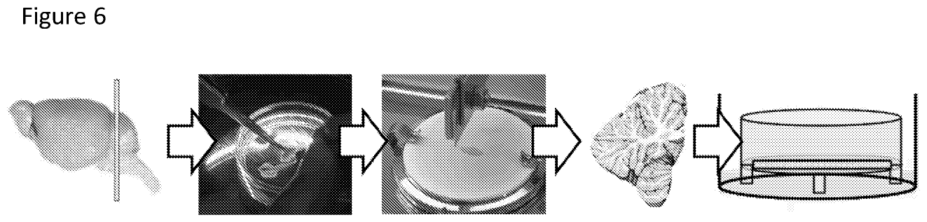

FIG. 6—Schematic transfection protocol for Rat Cerebellum slices. Experiment on postnatal day 8 (P8) rat pups. Cerebellum is dissected from the brain in ice-cold preparation medium using a stereomicroscope. 350 μm thick slices are made in the sagittal orientation using a Mcllwain tissue chopper or vibrotome. Millipore cell culture inserts (0.25 ml per well and 50 μl on top). Transfections started at 3-5 days in vitro (D3-5) by adding the transfection (100 μl) to the slice in top well.

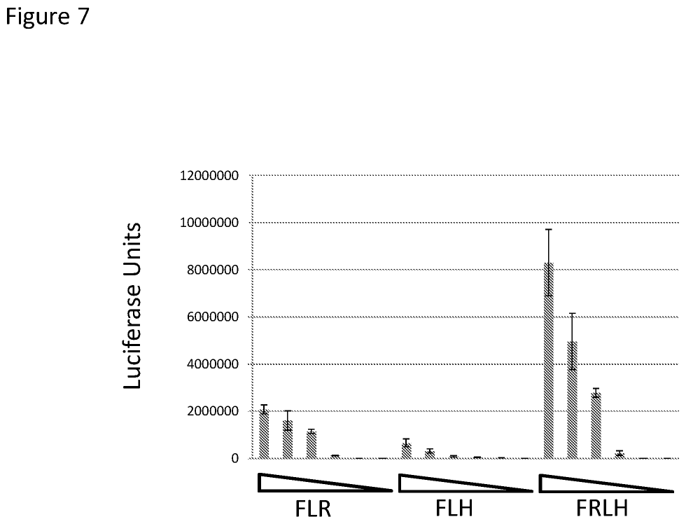

FIG. 7—Endosomal escape system on same peptide. Transfection of human skin fibroblasts with pCMV-gluc and FLR, FLH and FRLH with decreasing peptide:pDNA charge ratio (5:1, 4:1, 3:1, 2:1, 1:1). Gluc was measured on day 1 post-transfection, 1 ug pDNA was transfected in 12 well plates of 2×10⁵ cells. Bars are SD. N=3.

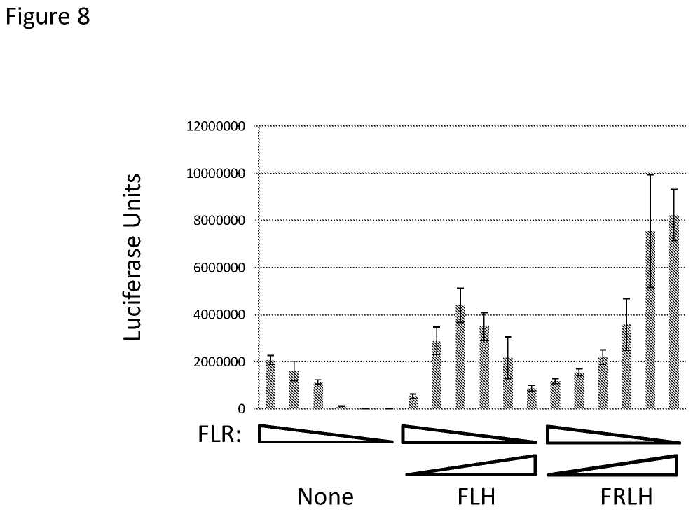

FIG. 8—Endosomal escape system on accessory peptide. Transfection of human skin fibroblasts with pCMV-gluc and FLR, with or without increasing proportions of FLH and FRLH (5:1, 4:1, 3:1, 2:1, 1:1 of FLR, with accessory peptides making up peptide charge to 5:1 against pDNA). Gluc was measured on day 1 post-transfection, 1 ug pDNA was transfected in 12 well plates of 2×10⁵ cells. Bars are SD. N=3.

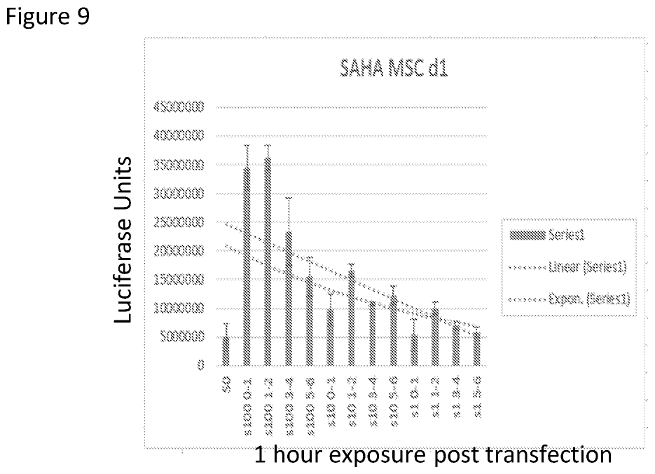

FIG. 9—HDACi activity is early in transfection (during macropinocytosis and endosomal trafficking) not later (affecting transcription activity). Transfection of human immortalised MSCs with pCMV-gluc and FLR:FLH (1:1) by rapid transfection (5 mins). Transfection was removed and exposed to SAHA (1-100 uM, S1-100) for 1 hour in the first 6 hours of uptake/transfection. Gluc was measured on day 1 post-transfection, 1 ug pDNA was transfected in 12 well plates of 2×10⁵ cells. Bars are SD. N=3.

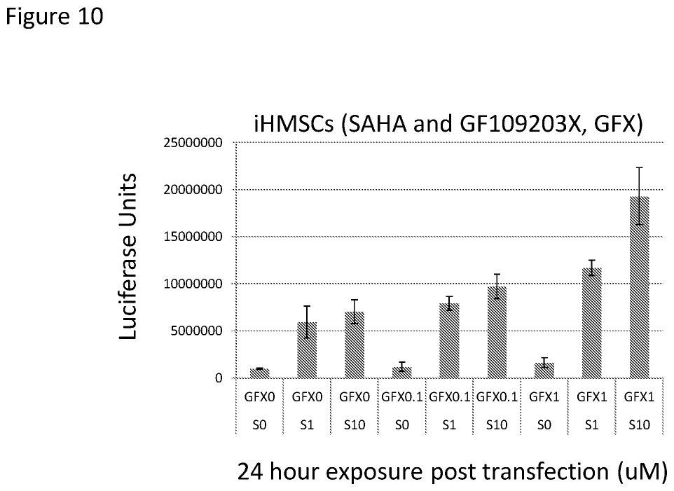

FIG. 10—Transfection of iHMSCs in the presence of HDAC and PKC inhibition (inhibitors SAHA and GF109203X, GFX). GFX0=0 μGF109203X, GFX0.1=0.1 μM GF109203X, GFX1=1 μM GF109203X, S0=0 μM SAHA, S1=1 μM SAHA, S10=10 μM SAHA.

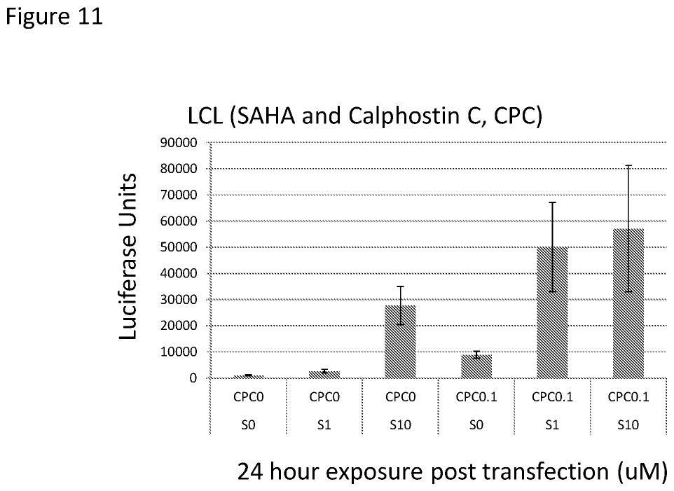

FIG. 11—Transfection of LCL in the presence of HDAC and PKC inhibition (inhibitors SAHA and Calphostin C). CPC0=0 μM Calphostin C, CPC0.1=0.1 μM Calphostin C, S0=0 μM SAHA, S1=1 μM SAHA, S10=10 μM SAHA.

### MATERIALS USED

pCMV-Gluc is a mammalian expression vector that expresses Gaussia luciferase under the control of a CMV promoter. It was obtained from New England Biolabs Inc. (NEB) https://international.neb.com/products/n8081-pcmv-gluc-2-control-plasmid#Product %20Information).

pGM (pGM206) is a CpG free DNA vector version expressing Gaussia luciferase (pG4-hCEFI-soGluc) (http://spiral.imperial.ac.uk:8080/bitstream/10044/1/19179/2/Biomaterials_32_10_2011.pdf).

The skilled person will recognise that any suitable control vector may be used to demonstrate transfection efficiency.

### Example Sequences for the Transfection Delivery Molecule

### Example HS-GAG Binding Sequences

P21 amino acid sequence
(SEQ ID NO. 1)
KRKKKGKGLGKKRDPCLRKYK
P21 nucleotide sequence (with a methione/ATG):
(SEQ ID NO: 2)
aagcgcaagaagaagggcaaaggcctgggcaagaagcgcgatccgtgcc
tgcgcaagtataag
PDGF (194-211) amino acid sequence:
(SEQ ID NO. 3)
GRPRESGKKRKRKRLKPT
PDGF (194-211) nucleotide sequence:
(SEQ ID NO: 4)
ggccgcccgcgcgaaagcggcaaaaaacgcaaacgcaaacgcctgaaac
cgacc
FGF7B amino acid sequence:
(SEQ ID NO. 5).
TYASAKWTHNGGEMFVALNQ
FGF7B nucleotide sequence:
(SEQ ID NO: 6)
Acctatgcgagcgcgaaatggacccataacggcggcgaaatgtttgtgg
cgctgaaccag
FGF2 HBD B(247-262) amino acid sequence:
(SEQ ID NO. 7).
TYRSRKYTSWYVALKR
FGF2 HBD B(247-262) nucleotide sequence:
(SEQ ID NO: 8)
acctatcgcagccgcaaatataccagctggtatgtggcgctgaaacgc

### Nucleotides Encoding 8R Protein Transduction Domain Sequence:

(SEQ ID NO: 9)
CGA AGA CGC AGG AGA CGT CGA AGG

### Example Delivery Molecule Nucleotide Sequence (P21-Cargo-8R):

(SEQ ID NO: 10)
aagcgcaagaagaagggcaaaggcctgggcaagaagcgcgatccgtgcc
tgcgcaagtataagNcgaagacgcaggagacgtcgaagg

N=cargo nucleic acid sequence of various length (i.e. the number of nucleotide residues may vary), or another molecular entity.

Two versions of each of the nanobody variants of the ScFv antibodies were made; one with identical sequence to the ScFv vHH domain (Frame domain1-CDR1-Frame domain 2-CDR2-Frame domain 3-CDR3-IgA Hinge domain/Frame domain 4) and one in which the CDR1, 2 and 3 domains were grafted into a generic vHH domain sequence. Both versions have comparable activity and the grafting version was created to prove that simply grafting the CDR domains onto a generic antibody also works.

Below are the sequences of the HS4C3, and AO4BO8 ScFv vHH and grafted vHH:

HS4C3 ScFv vHH
(SEQ ID NO: 11)
EVQLVESGGGLVQPRGSLRLSCAASGFTVSSNEMSWIRQAPGKGLEWVS
SISGGSTYYADSRKGRFTISRDNSKNTLYLQMNNLRAEGTAAYYCGRRL
KDPSTPPTPSPSTPPTPSPS
CDR1
GFTVSSNE
CDR2
ISGGST
CDR3
GRRLKD
HS4C3 ScFv vHH nucleotide sequence
(SEQ ID NO: 12)
gaagtgcagctggtggaaagcggcggcggcctggtgcagccgcgcggca
gcctgcgcctgagctgcgcggcgagcggctttaccgtgagcagcaacga
aatgagctggattcgccaggcgccgggcaaaggcctggaatgggtgagc
agcattagcggcggcagcacctattatgcggatagccgcaaaggccgct
ttaccattagccgcgataacagcaaaaacaccctgtatctgcagatgaa
caacctgcgcgcggaaggcaccgcggcgtattattgcggccgccgcctg
aaagatccgagcaccccgccgaccccgagcccgagcaccccgccgaccc
cgagcccgagc
HS4C3 grafted vHH
(SEQ ID NO: 13)
QVQLVESGGGSVQAGGSLRLSCTASGFTVSSNELGWFRQAPGQERWAVA
AISGGSTYYADSVKGRFTISRDNAKNTVTLQMNNLKPEDTAIYYCGRRL
KDWGQGTQVTVSSPSTPPTPSPSTPPTPSPS
CDR1
GFTVSSNE
CDR2
ISGGST
CDR3
GRRLKD
HS4C3 grafted vHH nucleotide
(SEQ ID NO: 14)
caggtgcagctggtggaaagcggcggcggcagcgtgcaggcgggcggca
gcctgcgcctgagctgcaccgcgagcggctttaccgtgagcagcaacga
actgggctggtttcgccaggcgccgggccaggaacgctgggcggtggcg
gcgattagcggcggcagcacctattatgcggatagcgtgaaaggccgct
ttaccattagccgcgataacgcgaaaaacaccgtgaccctgcagatgaa
caacctgaaaccggaagataccgcgatttattattgcggccgccgcctg
aaagattggggccagggcacccaggtgaccgtgagcagcccgagcaccc
cgccgaccccgagcccgagcaccccgccgaccccgagcccgagc
A04B08 ScFv vHH
(SEQ ID NO: 15)
EDQLVESGGGLVQPGGSLRPSCAASGFAFSSYALHWVRRAPGKGLEWVS
AIGTGGDTYYADSVMGRFTISRDNAKKSLYLHMNSLIAEDMAVYYCSLR
MNGWRAHQPSTPPTPSPSTPPTPSPS
CDR1
GFAFSSYA
CDR2
IGTGGDT
CDR3
SLRMNGWRAHQ
A04B08 ScFv vHH nucleotide sequence
(SEQ ID NO: 16)
gaagatcagctggtggaaagcggcggcggcctggtgcagccgggcggca
gcctgcgcccgagctgcgcggcgagcggctttgcgtttagcagctatgc
gctgcattgggtgcgccgcgcgccgggcaaaggcctggaatgggtgagc
gcgattggcaccggcggcgatacctattatgcggatagcgtgatgggcc
gctttaccattagccgcgataacgcgaaaaaaagcctgtatctgcatat
gaacagcctgattgcggaagatatggcggtgtattattgcagcctgcgc
atgaacggctggcgcgcgcatcagccgagcaccccgccgaccccgagcc
cgagcaccccgccgaccccgagcccgagc
A04B08 grafted vHH
(SEQ ID NO: 17)
QVQLVESGGGSVQAGGSLRLSCTASGFAFSSYALGWFRQAPGQERWAVA
AIGTGGDTYYADSVKGRFTISRDNAKNTVTLQMNNLKPEDTAIYYCSLR
MNGWRAHQWGQGTQVTVSSPSTPPTPSPSTPPTPSPS
CDR1
GFAFSSYA
CDR2
IGTGGDT
CDR3
SLRMNGWRAHQ
AO4B08 grafted vHH nucleotide sequence
(SEQ ID NO: 18)
caggtgcagctggtggaaagcggcggcggcagcgtgcaggcgggcggca
gcctgcgcctgagctgcaccgcgagcggctttgcgtttagcagctatgc
gctgggctggtttcgccaggcgccgggccaggaacgctgggcggtggcg
gcgattggcaccggcggcgatacctattatuggatagcgtgaaaggccg
ctttaccattagccgcgataacgcgaaaaacaccgtgaccctgcagatg
aacaacctgaaaccggaagataccgcgatttattattgcagcctgcgca
tgaacggctggcgcgcgcatcagtggggccagggcacccaggtgaccgt
gagcagcccgagcaccccgccgaccccgagcccgagcaccccgccgacc
ccgagcccgagc

1. A method of transfecting a cell with a molecule, the method comprising the steps of:
adding a molecule for transfection to the cells; and
modulating the activity of protein kinase C (PKC) in the cell and/or providing a pH responsive peptide comprising between about 5 and about 20 histidine residues.

2. The method according to claim 1, wherein the modulation of the activity of PKC in the cell comprises activation of PKC activity.

3. The method according to claim 1 or 2, wherein the modulation of PKC activity is provided by addition of a modulator of PKC selected from the group comprising phorbol 12-myristate 13-acetate (PMA), Ingenol 3-angelate (I3A) and bryostatin; or functional analogues and derivatives thereof; or the modulator of PKC is a genetic silencer siRNA arranged to inhibit expression of PKC.

4. The method according to any preceding claim, wherein the modulation of PKC activity is at a time of between about 1 and about 3 hours after starting transfection.

5. The method according to any preceding claim, further comprising the modulation of histone deacetylase (HDAC) in the cell.

6. The method according to claim 5, wherein modulation of HDAC activity is provided by addition of a HDAC inhibitor selected from the group comprising suberoylanilide hydroxamic acid (SAHA), panobinostat, trichostatin A (TSA), tubastatin A, and valproic acid; or functional analogues and derivatives thereof; or the HDAC modulator is a genetic silencer siRNA arranged to inhibit expression of HDAC.

7. The method according to any preceding claim, wherein PMA/TPA activation of PKC activity is provided in combination with one or more of HDAC inhibitors panobinostat, TSA or SAHA.

8. The method according to any preceding claim, wherein the molecule for transfection comprises or consists of nucleic acid, a protein, a peptide, or a nanoparticle.

9. The method according to claim 8, wherein the nucleic acid is DNA encoding one or more gene sequences and/or one or more regulatory sequences.

10. The method according to any preceding claim, wherein the molecule for transfection comprises a transcription factor, or a nucleic acid encoding a transcription factor.

11. The method according to any preceding claim, wherein the molecule for transfection comprises a growth factor or a nucleic acid encoding a growth factor.

12. The method according to any preceding claim, wherein the molecule for transfection is associated with, complexed with, entrapped within, or linked to, a transfection delivery molecule.

13. The method according to claim 12, wherein the transfection delivery molecule is selected from the group comprising a nucleic acid, a peptide, a protein, a viral particle, a virus-like particle, a non-viral molecule, a synthetic polymer, and a glycosaminoglycan (GAG)-binding enhanced transduction (GET)-cargo molecule, or a pH mediated variant thereof.

14. The method according to any preceding claim, wherein the cell or a population of cells for transfection is selected from the group comprising peripheral blood mononuclear cells (PBMCs), hematopoietic stem cells (HSCs), mesenchymal stem cells (MSCs), induced pluripotent stem cells (IPSCs), human embryonic stem cells (HESCs), sperm, oocytes, skeletal muscle cells, brain cells, and lung cells, or combinations thereof.

15. The method according to any preceding claim, wherein pH responsive peptide further comprises a sequence of between 10 and 25 K and/or L residues.

16. The method according to any preceding claim, wherein the pH responsive peptide comprises or consists of the sequence KLLKLLLKLLLKLLK(H₅₋₂₀).

17. A composition comprising:
a modulator of PKC and/or a pH responsive peptide comprising between about 5 and about 20 histidine residues; and
a molecule for transfection.

18. The composition according to claim 17, further comprising a HDAC modulator.

19. A kit comprising:
a modulator of PKC and/or a pH responsive peptide comprising between about 5 and about 20 histidine residues; and
a molecule for transfection.

20. The kit according to claim 19, further comprising a HDAC modulator.

21. Use of a modulator of PKC and/or a pH responsive peptide comprising between about 5 and about 20 histidine residues for increasing cell transfection efficiency, optionally, in combination with a HDAC modulator.

22. A method of gene therapy comprising administering to a subject:
(a) a modulator of PKC and/or a pH responsive peptide comprising between about 5 and about 20 histidine residues; and
(b) a molecule for transfection, wherein the molecule for transfection comprises nucleic acid.

23. A method of treatment of a subject comprising administering to the subject:
(a) a modulator of PKC and/or a pH responsive peptide comprising between about 5 and about 20 histidine residues; and
(b) a molecule for transfection, wherein the molecule for transfection comprises a therapeutically effective molecule.

24. A method of treatment of a subject comprising administering to the subject cells that have been transformed according the method of any one of claims 1 to 16.

25. A modulator of PKC and/or a pH responsive peptide comprising between about 5 and about 20 histidine residues for use in combination with a molecule for transfection as a medicament.


SEQUENCE LISTING (119 sequences)

SEQ ID NO: 1 - protein, 21 aa, Artificial Sequence
  Peptide
KRKKKGKGLGKKRDPCLRKYK

SEQ ID NO: 2 - DNA, 63 bp, Artificial Sequence
  Peptide
aagcgcaagaagaagggcaaaggcctgggcaagaagcgcgatccgtgcctgcgcaagtataag

SEQ ID NO: 3 - protein, 18 aa, Artificial Sequence
  Peptide
GRPRESGKKRKRKRLKPT

SEQ ID NO: 4 - DNA, 54 bp, Artificial Sequence
  Peptide
ggccgcccgcgcgaaagcggcaaaaaacgcaaacgcaaacgcctgaaaccgacc

SEQ ID NO: 5 - protein, 20 aa, Artificial Sequence
  Peptide
TYASAKWTHNGGEMFVALNQ

SEQ ID NO: 6 - DNA, 60 bp, Artificial Sequence
  Peptide
acctatgcgagcgcgaaatggacccataacggcggcgaaatgtttgtggcgctgaaccag

SEQ ID NO: 7 - protein, 16 aa, Artificial Sequence
  Peptide
TYRSRKYTSWYVALKR

SEQ ID NO: 8 - DNA, 48 bp, Artificial Sequence
  Peptide
acctatcgcagccgcaaatataccagctggtatgtggcgctgaaacgc

SEQ ID NO: 9 - DNA, 24 bp, Artificial Sequence
  Peptide
cgaagacgcaggagacgtcgaagg

SEQ ID NO: 10 - DNA, 63 bp, Artificial Sequence; identical to SEQ ID NO: 2
  Peptide

SEQ ID NO: 11 - DNA, 24 bp, Artificial Sequence
  Peptide
cgaagacgcaggagacgtcgaagg

SEQ ID NO: 12 - protein, 118 aa, Artificial Sequence
  ScFv
EVQLVESGGGLVQPRGSLRLSCAASGFTVSSNEMSWIRQAPGKGLEWVSSISGGSTYYADSRKGRFTISRDNSKNTLYLQMNNLRAEGTAAYYCGRRLKDPSTPPTPSPSTPPTPSPS

SEQ ID NO: 13 - protein, 8 aa, Artificial Sequence
  CDR
GFTVSSNE

SEQ ID NO: 14 - protein, 6 aa, Artificial Sequence
  CDR
ISGGST

SEQ ID NO: 15 - protein, 6 aa, Artificial Sequence
  CDR
GRRLKD

SEQ ID NO: 16 - DNA, 354 bp, Artificial Sequence
  ScFv
gaagtgcagctggtggaaagcggcggcggcctggtgcagccgcgcggcagcctgcgcctgagctgcgcggcgagcggctttaccgtgagcagcaacgaaatgagctggattcgccaggcgccgggcaaaggcctggaatgggtgagcagcattagcggcggcagcacctattatgcggatagccgcaaaggccgctttaccattagccgcgataacagcaaaaacaccctgtatctgcagatgaacaacctgcgcgcggaaggcaccgcggcgtattattgcggccgccgcctgaaagatccgagcaccccgccgaccccgagcccgagcaccccgccgaccccgagcccgagc

SEQ ID NO: 17 - protein, 129 aa, Artificial Sequence
  ScFv
QVQLVESGGGSVQAGGSLRLSCTASGFTVSSNELGWFRQAPGQERWAVAAISGGSTYYADSVKGRFTISRDNAKNTVTLQMNNLKPEDTAIYYCGRRLKDWGQGTQVTVSSPSTPPTPSPSTPPTPSPS

SEQ ID NO: 18 - protein, 8 aa, Artificial Sequence
  CDR
GFTVSSNE

SEQ ID NO: 19 - protein, 6 aa, Artificial Sequence
  CDR
ISGGST

SEQ ID NO: 20 - protein, 6 aa, Artificial Sequence
  CDR
GRRLKD

SEQ ID NO: 21 - DNA, 387 bp, Artificial Sequence
  ScFv
caggtgcagctggtggaaagcggcggcggcagcgtgcaggcgggcggcagcctgcgcctgagctgcaccgcgagcggctttaccgtgagcagcaacgaactgggctggtttcgccaggcgccgggccaggaacgctgggcggtggcggcgattagcggcggcagcacctattatgcggatagcgtgaaaggccgctttaccattagccgcgataacgcgaaaaacaccgtgaccctgcagatgaacaacctgaaaccggaagataccgcgatttattattgcggccgccgcctgaaagattggggccagggcacccaggtgaccgtgagcagcccgagcaccccgccgaccccgagcccgagcaccccgccgaccccgagcccgagc

SEQ ID NO: 22 - protein, 124 aa, Artificial Sequence
  ScFv
EDQLVESGGGLVQPGGSLRPSCAASGFAFSSYALHWVRRAPGKGLEWVSAIGTGGDTYYADSVMGRFTISRDNAKKSLYLHMNSLIAEDMAVYYCSLRMNGWRAHQPSTPPTPSPSTPPTPSPS

SEQ ID NO: 23 - protein, 8 aa, Artificial Sequence
  CDR
GFAFSSYA

SEQ ID NO: 24 - protein, 7 aa, Artificial Sequence
  CDR
IGTGGDT

SEQ ID NO: 25 - protein, 11 aa, Artificial Sequence
  CDR
SLRMNGWRAHQ

SEQ ID NO: 26 - DNA, 372 bp, Artificial Sequence
  ScFv
gaagatcagctggtggaaagcggcggcggcctggtgcagccgggcggcagcctgcgcccgagctgcgcggcgagcggctttgcgtttagcagctatgcgctgcattgggtgcgccgcgcgccgggcaaaggcctggaatgggtgagcgcgattggcaccggcggcgatacctattatgcggatagcgtgatgggccgctttaccattagccgcgataacgcgaaaaaaagcctgtatctgcatatgaacagcctgattgcggaagatatggcggtgtattattgcagcctgcgcatgaacggctggcgcgcgcatcagccgagcaccccgccgaccccgagcccgagcaccccgccgaccccgagcccgagc

SEQ ID NO: 27 - protein, 135 aa, Artificial Sequence
  ScFv
QVQLVESGGGSVQAGGSLRLSCTASGFAFSSYALGWFRQAPGQERWAVAAIGTGGDTYYADSVKGRFTISRDNAKNTVTLQMNNLKPEDTAIYYCSLRMNGWRAHQWGQGTQVTVSSPSTPPTPSPSTPPTPSPS

SEQ ID NO: 28 - protein, 8 aa, Artificial Sequence
  CDR
GFAFSSYA

SEQ ID NO: 29 - protein, 7 aa, Artificial Sequence
  CDR
IGTGGDT

SEQ ID NO: 30 - protein, 11 aa, Artificial Sequence
  CDR
SLRMNGWRAHQ

SEQ ID NO: 31 - DNA, 405 bp, Artificial Sequence
  ScFv
caggtgcagctggtggaaagcggcggcggcagcgtgcaggcgggcggcagcctgcgcctgagctgcaccgcgagcggctttgcgtttagcagctatgcgctgggctggtttcgccaggcgccgggccaggaacgctgggcggtggcggcgattggcaccggcggcgatacctattatgcggatagcgtgaaaggccgctttaccattagccgcgataacgcgaaaaacaccgtgaccctgcagatgaacaacctgaaaccggaagataccgcgatttattattgcagcctgcgcatgaacggctggcgcgcgcatcagtggggccagggcacccaggtgaccgtgagcagcccgagcaccccgccgaccccgagcccgagcaccccgccgaccccgagcccgagc

SEQ ID NO: 32 - protein, 15 aa, Artificial Sequence
  Peptide
KLLKLLLKLLLKLLK

SEQ ID NO: 33 - protein, 25 aa, Artificial Sequence
  Peptide
KLLKLLLKLLLKLLKHHHHHHHHHH

SEQ ID NO: 34 - protein, 20 aa, Artificial Sequence
  Peptide
KLLKLLLKLLLKLLKHHHHH

SEQ ID NO: 35 - protein, 21 aa, Artificial Sequence
  Peptide
KLLKLLLKLLLKLLKHHHHHH

SEQ ID NO: 36 - protein, 22 aa, Artificial Sequence
  Peptide
KLLKLLLKLLLKLLKHHHHHHH

SEQ ID NO: 37 - protein, 23 aa, Artificial Sequence
  Peptide
KLLKLLLKLLLKLLKHHHHHHHH

SEQ ID NO: 38 - protein, 24 aa, Artificial Sequence
  Peptide
KLLKLLLKLLLKLLKHHHHHHHHH

SEQ ID NO: 39 - protein, 25 aa, Artificial Sequence
  Peptide
KLLKLLLKLLLKLLKHHHHHHHHHH

SEQ ID NO: 40 - protein, 26 aa, Artificial Sequence
  Peptide
KLLKLLLKLLLKLLKHHHHHHHHHHH

SEQ ID NO: 41 - protein, 27 aa, Artificial Sequence
  Peptide
KLLKLLLKLLLKLLKHHHHHHHHHHHH

SEQ ID NO: 42 - protein, 28 aa, Artificial Sequence
  Peptide
KLLKLLLKLLLKLLKHHHHHHHHHHHHH

SEQ ID NO: 43 - protein, 29 aa, Artificial Sequence
  Peptide
KLLKLLLKLLLKLLKHHHHHHHHHHHHHH

SEQ ID NO: 44 - protein, 30 aa, Artificial Sequence
  Peptide
KLLKLLLKLLLKLLKHHHHHHHHHHHHHHH

SEQ ID NO: 45 - protein, 31 aa, Artificial Sequence
  Peptide
KLLKLLLKLLLKLLKHHHHHHHHHHHHHHHH

SEQ ID NO: 46 - protein, 32 aa, Artificial Sequence
  Peptide
KLLKLLLKLLLKLLKHHHHHHHHHHHHHHHHH

SEQ ID NO: 47 - protein, 33 aa, Artificial Sequence
  Peptide
KLLKLLLKLLLKLLKHHHHHHHHHHHHHHHHHH

SEQ ID NO: 48 - protein, 34 aa, Artificial Sequence
  Peptide
KLLKLLLKLLLKLLKHHHHHHHHHHHHHHHHHHH

SEQ ID NO: 49 - protein, 35 aa, Artificial Sequence
  Peptide
KLLKLLLKLLLKLLKHHHHHHHHHHHHHHHHHHHH

SEQ ID NO: 50 - protein, 44 aa, Artificial Sequence
  Peptide
FLHTYRSRKYTSWYVALKRKLLKLLLKLLLKLLKHHHHHHHHHH

SEQ ID NO: 51 - protein, 49 aa, Artificial Sequence
  Peptide
TYRSRKYTSWYVALKRKLLKLLLKLLLKLLKHHHHHHHHHHRRRRRRRR

SEQ ID NO: 52 - protein, 49 aa, Artificial Sequence
  Peptide
TYRSRKYTSWYVALKRKLLKLLLKLLLKLLKRRRRRRRRHHHHHHHHHH

SEQ ID NO: 53 - protein, 49 aa, Artificial Sequence
  Peptide
TYRSRKYTSWYVALKRRRRRRRRRKLLKLLLKLLLKLLKHHHHHHHHHH

SEQ ID NO: 54 - protein, 6 aa, Artificial Sequence
  CDR
GMRPRL

SEQ ID NO: 55 - protein, 11 aa, Artificial Sequence
  CDR
HAPLRNTRTNT

SEQ ID NO: 56 - protein, 6 aa, Artificial Sequence
  CDR
GSRSSR

SEQ ID NO: 57 - protein, 7 aa, Artificial Sequence
  CDR
GRTVGRN

SEQ ID NO: 58 - protein, 7 aa, Artificial Sequence
  CDR
GKVKLPN

SEQ ID NO: 59 - protein, 8 aa, Artificial Sequence
  CDR
SGRKGRMR

SEQ ID NO: 60 - protein, 11 aa, Artificial Sequence
  CDR
SLRMNGWRAHQ

SEQ ID NO: 61 - protein, 7 aa, Artificial Sequence
  CDR
RRYALDY

SEQ ID NO: 62 - protein, 7 aa, Artificial Sequence
  CDR
LKQQGIS

SEQ ID NO: 63 - protein, 12 aa, Artificial Sequence
  CDR
AMTQKKPRKLSL

SEQ ID NO: 64 - protein, 12 aa, Artificial Sequence
  CDR
SGRKYFRARDMN

SEQ ID NO: 65 - protein, 15 aa, Artificial Sequence
  Peptise
RQIKWFQNRRMKWKK

SEQ ID NO: 66 - protein, 11 aa, Artificial Sequence
  Peptide
YGRKKRRQRRR

SEQ ID NO: 67 - protein, 18 aa, Artificial Sequence
  Peptide
RGGRLSYSRRRFSTSTGR

SEQ ID NO: 68 - protein, 10 aa, Artificial Sequence
  Peptide
RRLSYSRRRF

SEQ ID NO: 69 - protein, 12 aa, Artificial Sequence
  Peptide
PIRRRKKLRRLK

SEQ ID NO: 70 - protein, 12 aa, Artificial Sequence
  Peptide
RRQRRTSKLMKR

SEQ ID NO: 71 - protein, 15 aa, Artificial Sequence
  Peptide
RRRRNRTRRNRRRVR

SEQ ID NO: 72 - protein, 19 aa, Artificial Sequence
  Peptide
KMTRAQRRAAARRNRWTAR

SEQ ID NO: 73 - protein, 13 aa, Artificial Sequence
  Peptide
TRRQRTRRARRNR

SEQ ID NO: 74 - protein, 13 aa, Artificial Sequence
  Peptide
GRKKRRQRRRPPQ

SEQ ID NO: 75 - protein, 13 aa, Artificial Sequence
  Peptide
GRRRRRRRRRPPQ

SEQ ID NO: 76 - protein, 27 aa, Artificial Sequence
  Peptide
GWTLNSAGYLLGKINLKALAALAKKIL

SEQ ID NO: 77 - protein, 17 aa, Artificial Sequence
  Peptide
KLALKLALKLALALKLA

SEQ ID NO: 78 - protein, 27 aa, Artificial Sequence
  Peptide
MGLGLHLLVLAAALQGAWSQPKKKRKV

SEQ ID NO: 79 - protein, 27 aa, Artificial Sequence
  Peptide
GALFLGWLGAAGSTMGAWSQPKKKRKV

SEQ ID NO: 80 - protein, 27 aa, Artificial Sequence
  Peptide
GALFLGFLGAAGSTMGAWSQPKKKRKV

SEQ ID NO: 81 - protein, 27 aa, Artificial Sequence
  Peptide
GALFLGFLGAAGSTMGAWSQPKSKRKV

SEQ ID NO: 82 - protein, 21 aa, Artificial Sequence
  Peptide
KETWWETWWTEWSQPKKKRKV

SEQ ID NO: 83 - protein, 21 aa, Artificial Sequence
  Peptide
KETWFETWFTEWSQPKKKRKV

SEQ ID NO: 84 - protein, 4 aa, Artificial Sequence
  Peptide
RRRR

SEQ ID NO: 85 - protein, 5 aa, Artificial Sequence
  Peptide
RRRRR

SEQ ID NO: 86 - protein, 6 aa, Artificial Sequence
  Peptide
RRRRRR

SEQ ID NO: 87 - protein, 7 aa, Artificial Sequence
  Peptide
RRRRRRR

SEQ ID NO: 88 - protein, 8 aa, Artificial Sequence
  Peptide
RRRRRRRR

SEQ ID NO: 89 - protein, 9 aa, Artificial Sequence
  Peptide
RRRRRRRRR

SEQ ID NO: 90 - protein, 10 aa, Artificial Sequence
  Peptide
RRRRRRRRRR

SEQ ID NO: 91 - protein, 11 aa, Artificial Sequence
  Peptide
RRRRRRRRRRR

SEQ ID NO: 92 - protein, 12 aa, Artificial Sequence
  Peptide
RRRRRRRRRRRR

SEQ ID NO: 93 - protein, 13 aa, Artificial Sequence
  Peptide
RRRRRRRRRRRRR

SEQ ID NO: 94 - protein, 14 aa, Artificial Sequence
  Peptide
RRRRRRRRRRRRRR

SEQ ID NO: 95 - protein, 15 aa, Artificial Sequence
  Peptide
RRRRRRRRRRRRRRR

SEQ ID NO: 96 - protein, 16 aa, Artificial Sequence
  Peptide
RRRRRRRRRRRRRRRR

SEQ ID NO: 97 - protein, 17 aa, Artificial Sequence
  Peptide
RRRRRRRRRRRRRRRRR

SEQ ID NO: 98 - protein, 4 aa, Artificial Sequence
  Peptide
KKKK

SEQ ID NO: 99 - protein, 5 aa, Artificial Sequence
  Peptide
KKKKK

SEQ ID NO: 100 - protein, 6 aa, Artificial Sequence
  Peptide
KKKKKK

SEQ ID NO: 101 - protein, 7 aa, Artificial Sequence
  Peptide
KKKKKKK

SEQ ID NO: 102 - protein, 8 aa, Artificial Sequence
  Peptide
KKKKKKKK

SEQ ID NO: 103 - protein, 9 aa, Artificial Sequence
  Peptide
KKKKKKKKK

SEQ ID NO: 104 - protein, 10 aa, Artificial Sequence
  Peptide
KKKKKKKKKK

SEQ ID NO: 105 - protein, 11 aa, Artificial Sequence
  Peptide
KKKKKKKKKKK

SEQ ID NO: 106 - protein, 12 aa, Artificial Sequence
  Peptide
KKKKKKKKKKKK

SEQ ID NO: 107 - protein, 13 aa, Artificial Sequence
  Peptide
KKKKKKKKKKKKK

SEQ ID NO: 108 - protein, 14 aa, Artificial Sequence
  Peptide
KKKKKKKKKKKKKK

SEQ ID NO: 109 - protein, 15 aa, Artificial Sequence
  Peptide
KKKKKKKKKKKKKKK

SEQ ID NO: 110 - protein, 16 aa, Artificial Sequence
  Peptide
KKKKKKKKKKKKKKKK

SEQ ID NO: 111 - protein, 17 aa, Artificial Sequence
  Peptide
KKKKKKKKKKKKKKKKK

SEQ ID NO: 112 - protein, 8 aa, Artificial Sequence
  Peptide
RGRRRRRR

SEQ ID NO: 113 - protein, 8 aa, Artificial Sequence
  Peptide
RMRRRRRR

SEQ ID NO: 114 - protein, 8 aa, Artificial Sequence
  Peptide
RTRRRRRR

SEQ ID NO: 115 - protein, 8 aa, Artificial Sequence
  Peptide
RSRRRRRR

SEQ ID NO: 116 - protein, 10 aa, Artificial Sequence
  Peptide
RRRRRRRRRR

SEQ ID NO: 117 - protein, 8 aa, Artificial Sequence
  Peptide
RARRRRRR

SEQ ID NO: 118 - protein, 7 aa, Artificial Sequence
  Peptide
RRRRRRR

SEQ ID NO: 119 - protein, 8 aa, Artificial Sequence
  Peptide
RRRRRRRR